(19) United States
(12) Patent Application Publication
(10) Pub. No.: US 2004/0235749 A1
(43) Pub. Date: Nov. 25, 2004

(54) G-protein coupled receptor antagonists

(76) Inventors: Sylvain Chemtob, Montreal (CA); Krishna Peri, Ville St-Laurent (CA)

Correspondence Address: PAUL, HASTINGS, JANOFSKY & WALKER LLP, P.O. BOX 919092, SAN DIEGO, CA, 92191-9092, US

(21) Appl. No.: 10/678,413

(22) Filed: Oct. 2, 2003

Related U.S. Application Data
(63) Continuation-in-part of application No. 09/787,334, filed on Jun. 19, 2001.

(30) Foreign Application Priority Data
Sep. 15, 1999 (WO) PCT/CA99/00844

(51) Int. Cl. A61K 38/10; A61K 38/08

(52) U.S. Cl. 514/16; 514/17; 514/15

(57) ABSTRACT

Methods of modulating the function of G-protein coupled receptors (GPCRs) using peptide antagonists of the GPCR are provided. The peptide antagonists are derived from the sequence of a juxtamembrane extracellular structural element of the target GPCR and selectively modulate the function of the receptor from which they are derived. The GPCR peptide antagonists include peptides comprising naturally occurring amino acids as well as peptide analogues, peptide derivatives, peptidomimetics and peptide variants. Methods of selecting the peptide antagonists are also provided. The peptides have therapeutic application in the treatment, amelioration or prophylaxis of diseases or conditions associated with changes in GPCR activity.

### FIELD OF THE INVENTION

The present invention pertains to the field of therapeutics, and in particular to the use of antagonists of G-protein coupled receptors as therapeutic compounds.

### BACKGROUND

G-protein coupled receptors (GPCRs) are membrane proteins that function as one part of a multi-component complex involved in signal transduction. The GPCRs are a growing superfamily comprising over 250 known glycoproteins in humans, with an estimated one thousand or more expected to be discovered in the near future.

GPCRs share a common signaling mechanism, whereby signal transduction across the membrane involves intracellular transducer elements known as G-proteins (named for their ability to bind and hydrolyze the nucleotide GTP). When a chemical messenger, or ligand, binds to a specific site on the extracellular surface of the receptor, the conformation of the receptor changes so that it can interact with and activate an intracellular G-protein. In general, activation of GPCRs by ligands will induce one of the following effector responses: activation of adenylyl cyclase, inhibition of adenylyl cyclase, or stimulation of phospholipase C activity. When the effector adenylyl cyclase is either activated or inhibited, it produces changes in the concentration of the molecule cyclic adenosine monophosphate (cAMP). Phospholipase C catalyses the cleavage of phosphatidylinositol-bisphosphate (PIP₂) into inositol triphosphate (IP₃) and diacylglycerol (DAG); IP₃ then causes calcium ions (Ca²⁺) to be released into the cytoplasm. cAMP and Ca²⁺ are termed second messengers. Alterations in cellular levels of second messengers act to alter the behaviour of other target proteins in the cell. Diverse mediators including hormones, neurotransmitters, catecholamines, chemokines, eicosanoids, olfactory and photo-sensory stimuli exert their effects by activating GPCRs.

Mammalian GPCRs can be divided into three broad groups: Class A receptors, which are related to rhodopsin, Class B receptors, which are related to calcitonin and Class C receptors, which are related to metabotropic glutamate receptors. Various orphan and unclassified GPCRs that do not fit into one of these groups are also known. The rhodopsin family is by far the largest class of GPCRs. Class A and B GPCRs comprise over 90% of known GPCRs and share many structural and functional similarities.

The generic structure of a GPCR consists of a N-terminal domain (of 7-595 amino acids), seven transmembrane (TM) helical segments (each 17-23 amino acids), three extracellular loops and three intracellular loops (each 5-230 amino acids) and a C-terminal segment (of 12-359 amino acids). The TM segments form alpha helices, as deduced from the electron density map of rhodopsin (Unger, V. M. et al. 1997), some of which may extend beyond the lipid bilayer. The lengths of some TM segments and loops thus may be uneven and dynamic. TM segments contain one or more proline and glycine residues and as a result, are kinked and tightly packed by hydrogen bonds and salt bridges; structural changes in the receptor initiated by binding of the ligand are transmitted by the TM core, resulting in G-protein dissociation and interactions with other signaling molecules.

Current drug discovery technologies focus on the ligand as the lead compound and generate targeted chemical libraries; these libraries are screened using a variety of technologies including radioligand binding, GTPγS-binding and reporter gene assays. Agonist or antagonist binding may not mimic exactly the interactions of the ligand with the receptor, but is effective in displacing the ligand in radioligand-binding assays, thus interfering with signal generation. Mass screening of random chemical libraries also yield compounds that interfere with ligand binding if radioligand binding and displacement are used as the outcome measures.

Peptide effectors based on the primary sequence of the second, third and fourth intracellular loops of GPCRs have been shown to be activators of G_{α} protein as evidenced by increased GTP binding and hydrolysis (Taylor, J. M. et al. 1994 Cell Signal. 6:841-9). Ectopic expression of the long peptides of the intracellular loops from β-adrenergic, muscarinic and adenosine receptors in cells have been shown to inhibit the function of the corresponding receptor specifically. The apparent discrepancy in the action of intracellular peptides which activate G_{α} proteins in in vitro assays and inhibit receptor-mediated signaling when ectopically expressed in cells, has not been resolved.

Peptides that hamper signal transduction from the prostaglandin F_{2α} receptor by binding to the intracellular molecular interface between the receptor and the G_{αq} or G_{60q} protein have been disclosed (see, U.S. Pat. No. 5,955,575). These peptides are between 15 and 50 amino acids in length and are derived from the third or fourth intracellular loop of the receptor, or from the N-terminal or C-terminal α-helices of a G_{α} protein.

A 21 amino acid peptide corresponding to the sequence of the TM domain of β₂-adrenoreceptor without sequences from the corresponding extracellular or intracellular loops has been shown to inhibit receptor dimer formation in vitro (Hebert, T. E. et al., 1996, J Biol Chem. 271:16384-92). As a result of the inability to form dimers, second messenger generation was compromised in cells over expressing the receptor and treated with the peptide.

Antagonists of the prostaglandin E2, subtype EP4, receptor that comprise an octapeptide sequence selected from the primary structure of the receptor and their use in improving glomerular filtration and/or urine output has been described (see U.S. patent application Ser. No. 20030017988). The region of the receptor primary structure from which the octapeptide sequence is taken is not discussed.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide G-protein coupled receptor antagonists. In accordance with one aspect of the present invention, there is provided a method of modulating the function of a G-protein coupled receptor (GPCR) in a mammal comprising administering to said mammal an effective amount of at least one GPCR antagonist, wherein said antagonist is a peptide, peptide derivative, peptide analogue or peptidomimetic compound comprising an amino acid sequence of about 5 to about 10 amino acids, said amino acid sequence having at least 70% identity to the sequence of a juxtamembrane extracellular region of said GPCR.

In accordance with another aspect of the present invention, there is provided a method of identifying a peptide antagonist of a mammalian GPCR comprising:

a) culturing cells in which the GPCR is expressed;

b) contacting the cells with a candidate compound, said candidate compound is a peptide, peptide derivative, peptide analogue or peptidomimetic compound comprising an amino acid sequence of about 5 to about 10 amino acids, said amino acid sequence having at least 70% identity to the sequence of a juxtamembrane extracellular region of said GPCR, and

c) measuring at least one cellular and/or physiological consequence of modulation of GPCR function in said cells,

wherein an increase or decrease in said cellular and/or physiological consequence indicates that the candidate compound is a peptide antagonist of the GPCR.

In accordance with another aspect of the present invention, there is provided a method of modulating a cellular or physiological process mediated by a mammalian G-protein coupled receptor (GPCR), said method comprising contacting cells expressing said GPCR with an effective amount of at least one GPCR antagonist, wherein said antagonist is a peptide, peptide derivative, peptide analogue or peptidomimetic compound comprising an amino acid sequence of about 5 to about 10 amino acids, said amino acid sequence having at least 70% identity to the sequence of a juxtamembrane extracellular region of said GPCR.

In accordance with one embodiment of the present invention, there is provided a G protein-coupled receptor antagonist which specifically binds to the juxtamembrane extracellular structural elements of the G protein-coupled receptor in a manner different from that of the natural ligand, and wherein said agonist or antagonist alters the transduction of an intracellular signal. The G protein-coupled receptor agonist or antagonist may be derived from the amino acid sequence of the receptor.

In accordance with an alternative embodiment of the present invention, there are provided G-protein coupled receptor antagonists comprising an amino acid sequence corresponding to the juxtamembrane extracellular region of a GPCR that interfere with the function of the GPCR.

In accordance with another embodiment of the present invention, the antagonist does not crossreact with other receptors and the antagonist is effective in the presence of excess ligand.

In accordance with another embodiment of the present invention, the antagonist comprises an amino acid sequence that corresponds to a sequence derived from one of the seven extracellular loops of a G-protein coupled receptor.

In accordance with another embodiment of the present invention, the antagonists comprise amino acid sequences derived from the first and second extracellular loops of prostanoid receptors. In accordance with this embodiment, the antagonist may include, without limitation, amino acid sequence of the FP receptor selected from the group consisting of ILGHRDYK (PCP-8; SEQ ID NO:1); WEDRFYLL (PCP-10; SEQ ID NO:2); YQDRFYLL (PCP-14; SEQ ID NO:3); ILAHRDYK (PCP-13.7; SEQ ID NO:4); ILGFRDYK (PCP-13.11; SEQ ID NO:5); ILGHKDYK (PCP-13.13; SEQ ID NO: 6); ILGHRNYK (PCP-13.14; SEQ ID NO:7); ILGHQDYK (PCP-13.18; SEQ ID NO:8); ILGHRDY-amide (PCP-13.20; SEQ ID NO:9); ILGHRDYK-amide (PCP-13.21; SEQ ID NO:1); ILGWRDYK (PCP-13.22; SEQ ID NO:10); ILGXRDYK (PCP-13.24; SEQ ID NO:11); SNVLCSIF(PCP-15; SEQ ID NO: 12), protein fusions and peptidomimetics thereof; wherein said amino acid sequence contains L and/or D-amino acids.

In accordance with a further embodiment of the present invention, there is provided a peptide having an amino acid sequence selected from the group consisting of SEQ ID NO:1 to 12 and wherein said amino acid sequence contains L- and/or D-amino acid, an amino acid sequence with at least about 90% homology to SEQ ID NO:1 to 12, and peptidomimetic thereof.

In accordance with another embodiment of the present invention, there is provided a pharmaceutical composition containing at least a G protein-coupled receptor agonist and antagonist of the present invention, mixture thereof, or functional derivatives thereof in association with a pharmaceutically acceptable carrier.

In accordance with another embodiment of the present invention, there is provided a method of identifying a compound as a G protein-coupled receptor antagonist capable of binding to the extracellular elements of the said receptor in a manner different from that of the natural ligand, comprising the steps of:

a) culturing cells which express said receptor or identifying animal tissues ex vivo or in vivo where physiological consequences are dependent on said receptor;

b) contacting said cells or tissues with said compound to be tested for antagonist activity at said receptor; and

c) measuring a response to alter the transduction of a signal resulting in physiological consequences selected from the group consisting of increments in cell calcium, phosphoinositide hydrolysis, increased/decreased cellular cyclic adenosine monophosphate, cell growth and/or differentiation, altered gene expression, and smooth muscle contraction or dilation, wherein said response is indicative of antagonist activity.

In accordance with an alternate embodiment of the invention, there is provided a method of testing a candidate peptide antagonist of a GPCR for its ability to modulate the function of the GPCR comprising the steps of:

1. culturing cells in which the target GPCR is expressed;

2. contacting the cells with the candidate peptide antagonist; and

3. measuring at least one cellular and/or physiological consequence of modulation of GPCR function.

### BRIEF DESCRIPTION OF THE FIGURES

FIG. 1 illustrates the inhibitory effects of PCP-8 and PCP-10 on FP receptor function upon stimulation with PGF_{2α} in accordance with the embodiment of the present invention;

FIG. 2A illustrates the effects of PCP-8 and PCP-10 on the diameter of the microvessels of pig retina upon stimulation with either PGF_{2α} or thromboxane A_{z} mimetic, U46619; 2B illustrates the dose response of PGF_{2α} on the diameter of pig microvessels treated previously with PCP-8 or PCP-10; 2C illustrates the effects of thromboxane A₂ mimetic, U46619, on the diameter of pig microvessels treated previously with PCP-8 and PCP-10;

FIG. 3A illustrates the effects of PCP-10 upon spontaneous contractions of uterine smooth muscle; 3B illustrates the dose response of prostaglandin F_{2α} in the presence/absence of PCP-8 and PCP-10 upon uterine smooth muscle contraction;

FIG. 4 illustrates the reversal of basal tone of bovine myometrium even in the presence of FP receptor ligand, PGF_{2α};

FIG. 5 illustrates the effect of SEQ ID NOs:78 and 79 on urotensin-induced vascular constriction;

FIGS. 6A and 6B illustrate the effect of SEQ ID NO:79 on inositol phosphate hydrolysis;

FIG. 7 illustrates the effect of SEQ ID NO:79 on somatostatin-14 contractile responses in porcine retinal microvessels;

FIG. 8 illustrates the effect of SEQ ID NOs:78 and 79 on urotensin-induced acute hypertension in prglet;

FIG. 9 illustrates the effect of SEQ ID NO: 79 on urotensin-induced acute constriction in upper bronchi;

FIG. 10 illustrates the effect of SEQ ID NOs:80, 81 and 82 on glucagon-induced hyperglycemia in rat;

FIG. 11 illustrates the effect of SEQ ID NO: 82 on glucagon-induced cAMP synthesis in rat primary hepatocytes;

FIGS. 12A and 12B illustrate the effect of SEQ ID NOs:83 and 84 on intraocular pressure in piglet;

FIG. 13 illustrates the efficacy of SEQ ID NO:84 in reversing experimentally induced ocular hypertension;

FIGS. 14A and 12B illustrate the effect of SEQ ID NOs:83 and 84 on latanoprost- and timolol-induced arteriole constriction;

FIGS. 15A and 15B illustrate the effect of SEQ ID NO:85 on vascular constriction;

FIGS. 16A, 16B and 16C illustrate the effect of SEQ ID NO:85 on c-PAF induced hypotension in piglet;

FIG. 17 illustrates the effect of SEQ ID NO:85 on sepsis-associated hypotension in piglet;

FIG. 18 illustrates the effect of SEQ ID NO:85 on PAF receptor induced inositol triphosphate synthesis in recombinant CHO cells;

FIG. 19 illustrates the effect of SEQ ID NO:86 on PTH(1-34) stimulated cAMP levels; and

FIG. 20 illustrates the effect of SEQ ID NOs:87, 88 and 89 on endothelin-induced vascular constriction.

### DETAILED DESCRIPTION OF THE INVENTION

The present invention provides for methods of modulating the function of G-protein coupled receptors (GPCRs) using peptide antagonists of the GPCR. The peptide antagonists are derived from the sequence of a juxtamembrane extracellular structural element of the target GPCR and selectively modulate the function of the receptor from which they are derived. In accordance with the present invention a “peptide” for use as a GPCR antagonist includes peptides comprising naturally occurring amino-acids as well as peptide analogues, peptide derivatives, peptidomimetics and peptide variants. Methods of selecting the peptide antagonists are also provided. The peptides have therapeutic application in the treatment, amelioration or prophylaxis of diseases or conditions associated with changes in GPCR activity.

In accordance with one embodiment of the present invention, there are provided G-protein coupled receptor antagonists that comprise an amino acid sequence corresponding to the juxtamembrane extracellular region of a GPCR that interfere with the function of the GPCR.

In another embodiment of the present invention, there is provided a new class of G-protein coupled receptor antagonists, which bind to the extracellular molecular surface, thus hampering signal transduction.

Also provided in another embodiment of the invention is a novel strategy to target the extracellular loops of the receptor which contribute to the structural or functional integrity of the receptor.

Antagonists may bind to cognate elements in the extracellular surface of the receptor and prevent the receptor function by interfering with its signal initiation or transduction. Alternatively, peptide antagonists may interfere with the function of a target receptor by other mechanisms.

Definitions

Naturally-occurring amino acids are identified throughout by the conventional three-letter or one-letter abbreviations indicated below, which are as generally accepted in the peptide art and are recommended by the IUPAC-IUB commission in biochemical nomenclature:

TABLE 1
Amino acid codes

| | 3-letter | 1-letter |
|---|---|---|
| Name | code | code |
| Alanine | Ala | A |
| Arginine | Arg | R |
| Asparagine | Asn | N |
| Aspartic | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The peptide sequences set out herein are written according to the generally accepted convention whereby the N-terminal amino acid is on the left and the C-terminal amino acid is on the right. By convention, L-amino acids are represented by upper case letters and D-amino acids by lower case letters.

Unless defined otherwise, all technical and scientific terms used herein have the same meaning as commonly understood by one of ordinary skill in the art to which this invention pertains.

The expression “a G protein-coupled receptor antagonist” is intended to mean any natural or synthetic compound, peptide, protein, antibody, peptidomimetic or small chemical molecule, without limitation, insofar as it can modulate the function of a target G protein-coupled receptor. In one embodiment, the antagonist specifically binds to the extracellular structural elements of the G protein-coupled receptor to alter transduction of a signal.

The term “GPCR peptide antagonist,” as used herein refers to a peptide of between about 5 and about 10 amino acids that is capable of modulating the function of a GPCR.

The expression “functional derivatives” of a G protein-coupled receptor antagonist is intended to mean mimetic compounds and/or structurally unrelated compounds with respect to the G protein-coupled receptor antagonist, which can specifically bind to the extracellular structural elements of the G protein-coupled receptor to alter transduction of a signal.

The terms “function” or “activity,” as used interchangeably herein with reference to a GPCR, refer to a cellular or physiological event that is directly or indirectly associated with a GPCR in vivo. Examples include, but are not limited to, GTP hydrolysis, changes in cellular calcium levels, phosphoinositide hydrolysis, changes in cellular cAMP levels, adenyl cyclase activation or inhibition, protein kinase A activity, phospholipase C activity, cell growth and/or differentiation, altered gene expression, smooth muscle contraction or dilation, vasoconstriction and dilation, nerve cell membrane potential, secretion from glandular cells, and the like.

The term “peptide,” as used herein, refers to a sequence of amino acid residues linked together by peptide bonds or by modified peptide bonds. The term “peptide” is intended to encompass peptide analogues, peptide derivatives, peptidomimetics and peptide variants.

The term “peptide analogue,” as used herein, refers to a peptide comprising one or more non-naturally occurring amino acid. Examples of non-naturally occurring amino acids include, but are not limited to, D-amino acids (i.e. an amino acid of an opposite chirality to the naturally occurring form), N-α-methyl amino acids, C-α-methyl amino acids, β-methyl amino acids, β-alanine (β-Ala), norvaline (Nva), norleucine (Nle), 4-aminobutyric acid (γ-Abu), 2-aminoisobutyric acid (Aib), 6-aminohexanoic acid (ε-Ahx), ornithine (orn), hydroxyproline (Hyp), sarcosine, citrulline, cysteic acid, cyclohexylalanine, α-amino isobutyric acid, t-butylglycine, t-butylalanine, 3-aminopropionic acid, 2,3-diaminopropionic acid (2,3-diaP), D- or L-phenylglycine, D- or L-2-naphthylalanine (2-Nal), 1,2,3,4-tetrahydroisoquinoline-3-carboxylic acid (Tic), D- or L-2-thienylalanine (Thi), D- or L-3-thienylalanine, D- or L-1-, 2-, 3- or 4-pyrenylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylalanine D- or L-p-methoxybiphenylalanine, methionine sulphoxide (MSO) and homoarginine (Har). Other examples include D- or L-2-indole(alkyl)alanines and D- or L-alkylalanines, wherein alkyl is substituted or unsubstituted methyl, ethyl, propyl, hexyl, butyl, pentyl, isopropyl, iso-butyl, or iso-pentyl, and phosphono- or sulfated (e.g. —SO₃H) non-carboxylate amino acids.

The term “peptide derivative,” as used herein, refers to a peptide comprising additional chemical or biochemical moieties not normally a part of a naturally occurring peptide. Peptide derivatives include peptides in which the amino-terminus and/or the carboxy-terminus and/or one or more amino acid side chain has been derivatised with a suitable chemical substituent group, as well as cyclic peptides, dual peptides, multimers of the peptides, peptides fused to other proteins or carriers, glycosylated peptides, phosphorylated peptides, peptides conjugated to lipophilic moieties (for example, caproyl, lauryl, stearoyl moieties) and peptides conjugated to an antibody or other biological ligand. Examples of chemical substituent groups that may be used to derivatise a peptide include, but are not limited to, alkyl, cycloalkyl and aryl groups; acyl groups, including alkanoyl and aroyl groups; esters; amides; halogens; hydroxyls; carbamyls, and the like. The substituent group may also be a blocking group such as Fmoc (fluorenylmethyl-O—CO—), carbobenzoxy (benzyl-O—CO—), monomethoxysuccinyl, naphthyl-NH—CO—, acetylamino-caproyl and adamantyl-NH—CO—. Other derivatives include C-terminal hydroxymethyl derivatives, O-modified derivatives (for example, C-terminal hydroxymethyl benzyl ether) and N-terminally modified derivatives including substituted amides such as alkylamides and hydrazides.

The term “peptidomimetic,” as used herein, refers to a compound that is structurally similar to a peptide and contains chemical moieties that mimic the function of the peptide. For example, if a peptide contains two charged chemical moieties having functional activity, a mimetic places two charged chemical moieties in a spatial orientation and constrained structure so that the charged chemical function is maintained in three-dimensional space. The term peptidomimetic thus is intended to include isosteres. The term “isostere,” as used herein, refers to a chemical structure that can be substituted for a peptide because the steric conformation of the chemical structure is similar, for example, the structure fits a binding site specific for the peptide. Examples of peptidomimetics include peptides comprising one or more backbone modifications (i.e. amide bond mimetics), which are well known in the art. Examples of amide bond mimetics include, but are not limited to, —CH₂NH—, —CH₂S—, —CH₂CH₂—, —CH═CH— (cis and trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J; Chem. Soc. Perkin Trans. I307-314 (1982); Almquist et al., J. Med Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)). Other examples of peptidomimetics include peptides substituted with one or more benzodiazepine molecules (see, for example, James, G. L. et al. (1993) Science 260:1937-1942) and peptides comprising backbones crosslinked to form lactams or other cyclic structures.

The term “variant peptide,” as used herein, refers to a peptide in which one or more amino acid residue has been deleted, added or substituted in comparison to the amino acid sequence of the juxtamembrane extracellular region of the GPCR to which the peptide corresponds. Typically, when a variant contains one or more amino acid substitutions they are “conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group. In accordance with the invention, a variant peptide comprises an amino acid sequence that is at least about 70% identical to the sequence of the juxtamembrane extracellular region of the target GPCR. In one embodiment, the variant peptides comprise an amino acid sequence that is at least about 80% identical to the sequence of the juxtamembrane extracellular region of the target GPCR. In another embodiment, the variant peptides comprise an amino acid sequence that is at least about 90% identical to the sequence of the juxtamembrane extracellular region of the target GPCR.

The term “percent (%) amino acid sequence identity,” as used herein with respect to a reference polypeptide is defined as the percentage of amino acid residues in a candidate peptide sequence that are identical with the amino acid residues in the reference polypeptide sequence after aligning the sequences and introducing gaps, if necessary, to achieve the maximum percent sequence identity, without considering any conservative substitutions as part of the sequence identity. Alignment for the purpose of determining percent amino acid sequence identity can be achieved in by various techniques known in the art, for instance, using publicly available computer software such as ALIGN or Megalign (DNASTAR). Those skilled in the art can determine appropriate parameters for measuring alignment, including any algorithms needed to achieve maximal alignment over the full length of the peptide sequence being used in the comparison.

The term “alkyl,” as used herein, refers to a straight chain, branched or cyclic alkyl group of one to ten carbon atoms. This term is exemplified by such groups as methyl, ethyl, n-propyl, i-propyl, n-butyl, t-butyl, 1-butyl (or 2-methylpropyl), cyclopropylmethyl, i-amyl, n-amyl, hexyl and the like.

The term “heteroalkyl,” as used herein, refers to an alkyl group wherein at least one carbon is replaced with a hetero atom, such as N, O or S.

The term “aryl,” as used herein, refers to an aromatic carbocyclic group having at least one aromatic ring (e.g., phenyl, tolyl, xylyl or biphenyl) or multiple condensed rings in which at least one ring is aromatic, (e.g., 1,2,3,4-tetrahydronaphthyl, naphthyl, anthryl, or phenanthryl, 9-fluorenyl etc.).

The term “heterocyclic,” as used herein, refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl, indanyl or benzo[b]thienyl) and having at least one hetero atom, such as N, O or S, within the ring.

The term “heteroaromatic,” as used herein, refers to a heterocycle in which at least one heterocyclic ring is aromatic.

The term “about,’ as used herein, refers to a +/−10% variation from the nominal value. It is to be understood that such a variation is always included in any given value provided herein, whether or not it is specifically referred to.

The terms “therapy,” and “treatment,” as used interchangeably herein, refer to an intervention performed with the intention of alleviating the symptoms associated with, preventing the development of, or altering the pathology of a disease, disorder or condition. Thus, the terms therapy and treatment are used in the broadest sense, and include the prevention (prophylaxis), moderation, reduction, and curing of a disease, disorder or condition at various stages. Those in need of therapy/treatment include those already having the disease, disorder or condition as well as those prone to, or at risk of developing, the disease, disorder or condition and those in whom the disease, disorder or condition is to be prevented.

Administration of the antagonists of the invention “in combination with” one or more further therapeutic agents, is intended to include simultaneous (concurrent) administration and consecutive administration. Consecutive administration is intended to encompass various sequences of administration of the therapeutic agent(s) and the antagonist(s) of the invention to the subject.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference).

### Target G-Protein Coupled Receptors

The strategy provided herein of targeting peptide antagonists to the juxtamembrane extracellular region of a GPCR is broadly applicable to a wide variety of mammalian GPCRs. Thus, the present invention provides for methods of modulating the function of a wide variety of GPCRs using peptides that correspond to the juxtamembrane extracellular region of the target GPCR. Target GPCRs contemplated by the present invention include Class A rhodopsin-like receptors, Class B secretin/calcitonin-like receptors and Class C metabotropic glutamate-like receptors as well as of various orphan, putative and unclassified GPCRs.

Examples of known Class A GPCRs include, but are not limited to, amide receptors (such as acetylcholine (muscarinic) receptors; adrenoceptors; dopamine receptors; histamine receptors; serotonin receptors; octopamine receptors and trace amine receptors); peptide receptors (such as angiotensin receptors; bombesin receptors; bradykinin receptors; C5a anaphylatoxin receptors; Fmet-leu-phe receptors; interleukin receptors; chemokine receptors; C-X-C chemokine receptors; BONZO receptors (CXC6R); C-X3-C chemokine receptors; XC chemokine receptors; CCK receptors; endothelin receptors; melanocortin receptors; melanocyte stimulating hormone receptors; adrenocorticotropic hormone receptors; melanocortin hormone receptors; neuropeptide Y receptors; neurotensin receptors; opioid receptors; somatostatin receptors; tachykinin receptors; substance P receptors; substance K receptors; neuromedin K receptors; vasopressin receptors; oxytocin receptors; conopressin receptors; galanin receptors; allatostatin receptors; GPCR 54 receptors; thrombin receptors; proteinase-activated receptors; orexin receptors; neuropeptide FF receptors; urotensin II receptors; adrenomedullin (G10D); GPR37/endothelin B-like receptors; neuromedin U receptors; PRXamide receptors and allatostatin C/drostatin C receptors); hormone protein receptors (such as follicle stimulating hormone (FSH) receptors; lutropin-choriogonadotropic hormone receptors; thyrotropin receptors; and gonadotropin receptors); rhodopsin/opsin receptors; olfactory receptors; prostanoid receptors (such as prostaglandin receptors (including prostaglandin E2 subtype EP1; prostaglandin E2/D2 subtype EP2; prostaglandin E2 subtype EP3; prostaglandin E2 subtype EP4 and prostaglandin F2-alpha); prostacyclin receptors and thromboxane receptors); nucleotide-like receptors (such as adenosine receptors and purinoceptors); cannabinoid receptors; platelet activating factor (PAF) receptors; gonadotropin-releasing hormone receptors; thyrotropin-releasing hormone receptors; secretagogue receptors; melatonin receptors; lysosphingolipid and LPA (EDG) receptors; leukotriene B4 receptors and Class A orphan receptors (such as platelet ADP and KI01 receptors and mas proto-oncogene receptors).

Examples of known Class B GPCRs include, but are not limited to, calcitonin receptors; corticotropin releasing factor receptors; gastric inhibitory peptide receptors; glucagon receptors; growth hormone-releasing hormone receptors; parathyroid hormone (PTH) receptors; secretin receptors; vasoactive intestinal polypeptide receptors; diuretic hormone receptors; latrophilin receptors; brain-specific angiogenesis inhibitor (BAI) receptors; methuselah-like proteins (MTH) receptors; cadherin EGF LAG (CELSR) receptors.

Examples of known Class C GPCRs include, but are not limited to, metabotropic glutamate receptors; extracellular calcium-sensing receptors; GABA-B receptors; orphan GPRC5 receptor; orphan GPCR6 receptor; bride of sevenless protein (BOSS) receptors and taste receptors (T1R).

Also contemplated are various putative, orphan and unclassified GPCRs, examples of which include, but are not limited to, ocular albinism protein receptors; Frizzled/Smoothened family receptors; vomeronasal receptors (V1R and V3R) receptors and R14 receptors.

In addition, it will be readily understood by those skilled in the art that the strategy provided by the present invention can be extended to those GPCRs identified in the future in order to provide for peptide antagonists to these new receptors and the use of the peptides to modulate receptor function.

One embodiment of the present invention provides for peptide antagonists to Class A peptide, amide, prostanoid, nucleotide-like and PAF receptors, Class B GPCRs, Class C GPCRs and various orphan GPCRs. Another embodiment provides for peptide antagonists to Class A peptide, prostanoid and PAF receptors, Class B receptors and orphan GPCRs. A further embodiment provides for peptide antagonists to Class A peptide and PAF receptors, Class B receptors and orphan GPCRs.

### Candidate Peptide Antagonists

In accordance with the present invention, the peptide antagonists comprise an amino acid sequence of about 5 to about 10 amino acids corresponding to the sequence of a juxtamembrane extracellular region of a GPCR. By “corresponding to” it is meant that the amino acid sequence comprised by the antagonist has at least about 70% sequence identity with a juxtamembrane extracellular region of the target GPCR. The peptide antagonist may comprise D- or L- amino acids or combinations thereof. Various peptide derivatives, antagonists and peptidomimetics are also contemplated as outlined above.

The sequence of the peptide may run in the same direction as that of the corresponding sequence in the GPCR (i.e. the N-terminus of the peptide corresponds to the N-terminal end of the corresponding amino acid sequence in the receptor), or the sequence of the peptide may be inverted (i.e. the N-terminus of the peptide corresponds to the C-terminal end of the corresponding amino acid sequence in the receptor). For example, for a juxtamembrane extracellular region having a sequence from N- to C-terminus: VAGCRVAA, the sequence of an inverted (“retro”) peptide corresponding to this region would be from N- to C-terminus: AAVRCGAV.

As is known in the art, the superfamily of G-protein coupled receptors has been characterized as having seven transmembrane domains (domains 1 through 7) linked by extracellular and intracellular loops. The peptides of the present invention correspond to one of the juxtamembrane regions of an extracellular loop of the protein, i.e. where an extracellular loop joins a transmembrane domain. These juxtamembrane extracellular regions occur both where an extracellular loop enters the membrane to become a transmembrane domain and where a transmembrane domain exits the membrane into the extracellular space to create an extracellular loop. A worker skilled in the art will appreciate that the transmembrane domains of receptor proteins are not rigidly defined but exhibit a certain fluidity and that, therefore, the juxtamembrane extracellular region is not a static point corresponding to a particular amino acid, but rather is a dynamic region comprising several amino acids, typically about 10 amino acids. Thus it will be understood that, due to the fluidity of the juxtamembrane extracellular region, more than one peptide may be designed that corresponds to this region. Each of the peptides of the invention correspond to a sequence that, in general, is partially within the membrane and partially in the extracellular space, however, this region may also at times be situated either entirely within the membrane or entirely in the extracellular space.

As indicated above, the peptide antagonists of the present invention include peptide analogues, peptide derivatives, peptidomimetics and peptide variants. Such compounds are well known in the art and may have significant advantages over naturally occurring peptides, including, for example, greater chemical stability, increased resistance to proteolytic degradation, enhanced pharmacological properties (such as, half-life, absorption, potency and efficacy), altered specificity (for example, a broad-spectrum of biological activities) and/or reduced antigenicity.

Examples of GPCRs against which peptide antagonists can be designed are provided above. Amino acid sequences for various mammalian GPCRs can be found in publicly available databases, such as SWISS-PROT, EMBL and the GenBank database maintained by the National Center for Biotechnology Information (NCBI). The GPCRDB, GRAP mutant database also contains information on the amino acid sequence of GPCRs. The GenBank Accession numbers for the amino acid sequences of representative examples of known human GPCRs are provided in Table 1. Alternatively, amino acid sequences for new or unknown GPCRs can be determined by standard gene cloning and sequencing techniques.

TABLE 1
GenBank Accession Numbers for Amino Acid Sequences of Representative Human GPCRs

| | |
|---|---|
| Accession | |
| Number | Receptor Name |

Acetylcholine (muscarinic) receptors

| | |
|---|---|
| P11229 | Muscarinic acetylcholine m1 [CHRM1] |
| P08172 | Muscarinic acetylcholine m2 [CHRM2] |
| P20309 | Muscarinic acetylcholine m3 [CHRM3] |
| P08173 | Muscarinic acetylcholine m4 [CHRM4] |
| P08912 | Muscarinic acetylcholine m5 [CHRM5] |

Adenosine and adenine nucleotide receptors

| | |
|---|---|
| P30542 | Adenosine a1 [ADORA1] |
| P29274 | Adenosine a2a [ADORA2A] |
| P29275 | Adenosine a2b [ADORA2B] |
| P33765 | Adenosine a3 [ADORA3] |
| P41231 | P2U purinoceptor 1 (ATP receptor) (P2U1) [P2RY2; P2RU1] |
| P47900 | P2Y purinoceptor 1 (ATP receptor) (P2Y1) [P2RY1] |
| P51582 | Uridine nucleotide receptor (UNR) (P2P) (P2Y4) [P2RY4] |
| P43657 | P2Y purinoceptor 5 (P2Y5) (RB intron encoded) [P2RY5] |
| Q15077 | P2Y purinoceptor 6 (P2Y6) [P2RY6] |
| Q15722 | P2Y purinoceptor 7 (P2Y7) (leukotriene B4 (chemoattractant receptor-like 1) |
| | [P2RY7; GPR16; CMKRL1] |
| Q99677 | P2Y purinoceptor 9 (P2Y9) [P2RY9] |

Adrenergic receptors

| | |
|---|---|
| P35348 | α-1A adrenergic (α-1C) [ADRA1A; ADRA1C] |
| P35368 | α-1B adrenergic [ADRA1B] |
| P25100 | α-1D adrenergic (α-1A) [ADRA1D; ADRA1A] |
| P08913 | α-2A adrenergic (SUBTYPE C10) [ADRA2A; ADRA2R; ADRAR] |
| P18089 | α-2B adrenergic (SUBTYPE C2) [ADRA2B] |
| P18825 | α-2C-1 adrenergic (SUBTYPE C4) [ADRA2C] |
| P35369 | α-2C-2 adrenergic |
| P08588 | β-1 adrenergic [ADRB1; ADRB1R; B1AR] |
| P07550 | β-2 adrenergic [ADRB2; ADRB2R; B2AR] |
| P13945 | β-3 adrenergic [ADRB3; ADRB3R; B3AR] |

Angiotensin receptors

| | |
|---|---|
| P30556 | Type-1a angiotensin II (AT1A) [AGTR1A; AGTR1; AT2R1] |
| Q13725 | Type-1b angiotensin II (AT1A) [AGTR1B; AT2R1B] |
| P50052 | Type-2 angiotensin II RECEPTOR (AT2) [AGTR2] |

Bombesin receptors

| | |
|---|---|
| P28336 | Neuromedin-B (NMB-R) (neuromedin-B-preferring bombesin) [NMBR] |
| P30550 | Gastrin-releasing peptide (GRP-R) (GRP-preferring bombesin) [GRPR] |
| P32247 | Bombesin subtype-3 (BRS-3) [BRS3] |

Bradykinin receptors

| | |
|---|---|
| P46663 | B1 bradykinin (BK-1R) [BDKRB1] |
| P30411 | B2 bradykinin (BK-2R) [BDKRB2; BKR2] |

Cannabinoid receptors

| | |
|---|---|
| P21554 | Cannabinoid 1 (CB1) (CB-R) [CNR1; CNR] |
| P34972 | Cannabinoid 2 (CB2) (CX5) [CNR2] |

Chemokine and chemotactic factor receptors

| | |
|---|---|
| Q16581 | C3A anaphylatoxin chemotactic (C3A-R) (C3AR) [C3AR1; C3R1; AZ3B; HNFAG09] |
| P21730 | C5A anaphylatoxin chemotactic (C5A-R) (CD88) [C5R1; C5AR] |
| P21462 | FMET-LEU-PHE (FMLP-R) (N-formyl peptide) (FPR) [FPR1] |
| P25090 | FMLP-related I (FMLP-R-I) (lipoxin A4) [FPRL1; FPRH2; FPR2; LXA4R] |
| P25089 | FMLP-related II (FMLP-R-II) [FPRL2; FPRH1] |
| P25024 | High affinity interleukin-8 A (IL-8R A) (CXCR-1) (CDW128) [IL8RA; CXCR1] |
| P25025 | High affinity interleukin-8 B (IL-8R B) (CXCR-2) [IL8RB; CXCR2] |
| P49682 | C-X-C chemokine receptor type 3 (CXC-R3) (CXCR-3) [CXCR3; GPR9] |
| P30991 | C-X-C chemokine receptor type 4 (CXC-R4) (CXCR-4) (SDF-1 receptor) |
| | (stromal cell-derived factor 1) (FUSIN) (CXCR-4) (LESTR) (LCR1) (FB22) |
| | (NPYRL) [CXCR4] |
| P32246 | C-C chemokine receptor type 1 (C-C CKR-1) (CCR-1) [CMKBR1; CMKR1] |
| P41597 | C-C chemokine receptor type 2 (C-C CKR-2) (CCR-2) (MCP-1-R) [CMKBR2] |
| P51677 | C-C chemokine receptor type 3 (C-C CKR-3) (CCR-3) [CMKBR3] |
| P51679 | C-C chemokine receptor type 4 (C-C CKR-4) (CCR-4) [CMKBR4] |
| P51681 | C-C chemokine receptor type 5 (C-C CKR-5) (CCR-5) [CMKBR5] |
| P51684 | C-C chemokine receptor type 6 (C-C CKR-6) (CCR-6) (GPR-CY4) |
| | (GPRCY4) (chemokine receptor-like 3) (CKR-L3) [CMKBR6; STRL22; GPR29; |
| | CKRL3] |
| P32248 | C-C chemokine receptor type 7 (C-C CKR-7) (CCR-7) (EBV-induced G protein- |
| | coupled receptor 1) (EBI1) (BLR2) [CMKBR7; EBI1; EVI1] |
| P51685 | C-C chemokine receptor type 8 (C-C CKR-8) (CCR-8) (GPR-CY6) (GPRCY6) |
| | (chemokine receptor-like 1) (CKR-L1) (TER1) [CMKBR8; CKRL1] |
| O00574 | G protein-coupled receptor BONZO [BONZO; STRL33; TYMSTR] |

Cholecystokinin/gastrin receptors

| | |
|---|---|
| P32238 | Cholecystokinin type A (CCK-A-R) [CCKAR; CCKRA] |
| P32239 | Gastrin/cholecystokinin type B (CCK-B-R) [CCKBR; CCKRB] |

Dopamine receptors

| | |
|---|---|
| P21728 | D(1A) dopamine [DRD1] |
| P21918 | D(1B) dopamine (D(5) dopamine) [DRD5; DRD1B] |
| P14416 | D(2) dopamine [DRD2] |
| P35462 | D(3) dopamine [DRD3] |
| P21917 | D(4) dopamine (D(2C) dopamine) [DRD4] |

Endothelin receptors

| | |
|---|---|
| P25101 | Endothelin-1 (ET-A) [EDNRA; ETRA] |
| P24530 | Endothelin B (ET-B) (endothelin non-selective type) [EDNRB; ETRB] |

Glycoprotein hormone receptors

| | |
|---|---|
| P23945 | Follicle stimulating hormone (FSH-R) [FSHR] |
| P22888 | Lutropin-choriogonadotropic hormone (LH/CG-R) (LSH-R) [LHCGR; LHRHR; LGCR] |
| P16473 | Thyrotropin (TSH-R) [TSHR] |

Histamine receptors

| | |
|---|---|
| P35367 | Histamine H1 [HRH1] |
| P25021 | Histamine H2 (gastric receptor I) [HRH2] |

Melanocortin receptors

| | |
|---|---|
| Q01718 | Adrenocorticotropic hormone (ACTH-R) (MC2-R) [MC2R; ACTHR] |
| Q01726 | Melanocyte stimulating hormone (MSH-R) [MC1R; MSHR] |
| P41968 | Melanocortin-3 (MC3-R) [MC3R] |
| P32245 | Melanocortin-4 (MC4-R) [MC4R] |
| P33032 | Melanocortin-5 (MC5-R) (MC-2) [MC5R] |

Melanotonin receptors

| | |
|---|---|
| P48039 | Melatonin type 1A (MEL-1A-R) [MTNR1A] |
| P49286 | Melatonin type IB (MEL-1B-R) [MTNR1B] |
| Q13585 | Melatonin-related [GPR50] |

Neuropeptide Y receptors

| | |
|---|---|
| P25929 | Neuropeptide Y type 1 (NPY1-R) [NPY1R; NPYR; NPYY1] |
| Q15761 | Neuropeptide Y type 5 (NPY5-R) [NPY5R; NPYR5] |
| P49146 | Neuropeptide Y type 2 (NPY2-R) [NPY2R] |
| P50391 | Neuropeptide Y type 4 (NPY4-R) [PPYR1; NPY4R] |

Neurotensin receptors

| | |
|---|---|
| P30989 | Neurotensin type 1 (NT-R-1) [NTSR1; NTRR] |

Opioid peptide receptors

| | |
|---|---|
| P41143 | δ-type opioid (DOR-1) [OPRD1; OPRD] |
| P41145 | κ-type opioid (KOR-1) [OPKR1; OPKR] |
| P35372 | μ-type opioid (MOR-1) [OPRM1; MOR1] |
| P41146 | Nociceptin (orphanin FQ) (κ-TYPE 3 OPIOID) (KOR-3) [OPRL1; ORL1; OOR] |

Orexin receptors

| | |
|---|---|
| O43613 | Orexin type 1 (OX1R) (hypocretin type 1) [HCRTR1] |
| O43614 | Orexin type 2 (OX2R) (hypocretin type 2) [HCRTR2] |

Platelet activating factor receptors

| | |
|---|---|
| P25105 | Platelet activating factor (PAF-R) [PTAFR; PAFR] |

Prostanoid receptors

| | |
|---|---|
| Q13258 | Prostaglandin D2 (prostanoid DP) [PTGDR] |
| P34995 | Prostaglandin E2, EP1 subtype (prostanoid EP1) [PTGER1] |
| P43116 | Prostaglandin E2, EP2 subtype (prostanoid EP2) [PTGER2] |
| P43115 | Prostaglandin E2, EP3 subtype (prostanoid EP3) [PTGER3] |
| P35408 | Prostaglandin E2, EP4 subtype (prostanoid EP4) [PTGER4; PTGER2] |
| P43088 | Prostaglandin F2-ALPHA (prostanoid FP) [PTGFR] |
| P43119 | Prostacyclin (prostanoid IP) [PTGIR; PRIPR] |
| P21731 | Thromboxane A2 (TXA2-R) (prostanoid TP) [TBXA2R] |

Releasing hormone receptors

| | |
|---|---|
| P30968 | Gonadotropin-releasing hormone (GRH-R) [GNRHR] |
| P34981 | Thyrotropin-releasing hormone (TRH-R) [TRHR] |
| Q95847 | Growth hormone secretagogue type 1 [GHSR] |

Serotonin receptors

| | |
|---|---|
| P08908 | 5-hydroxytryptamine 1A (5-HT-1A) [HTR1A] |
| P28222 | 5-hydroxytryptamine 1B (5-HT-1B) (5-HT-1D-BETA) (S12) [HTR1B; |
| | HTR1DB] |
| P28221 | 5-hydroxytryptamine 1D (5-HT-1D) (5-HT-1D-ALPHA) [HTR1D; HTR1DA] |
| P28566 | 5-hydroxytryptamine 1E (5-HT-1E) (S31) [HTR1E] |
| P30939 | 5-hydroxytryptamine 1F (5-HT-1F) [HTR1F] |
| P28223 | 5-hydroxytryptamine 2A (5-HT-2A) [HTR2A; HTR2] |
| P41595 | 5-hydroxytryptamine 2B (5-HT-2B) [HTR2B] |
| P28335 | 5-hydroxytryptamine 2C (5-HT-2C) (5-HT-1C) [HTR1C] |
| Q13629 | 5-hydroxytryptamine 4 (5-HT-4) [HTR4] (Fragment) |
| P47898 | 5-hydroxytryptamine 5A (5-HT-5A) [HTR5A] |
| P50406 | 5-hydroxytryptamine 6 (5-HT-6) [HTR6] |

Somatostatin receptors

| | |
|---|---|
| P30872 | Somatostatin type 1 (SS1R) (SRJF-2) [SSTR1] |
| P30874 | Somatostatin type 2 (SS2R) (SRIF-1) [SSTR2] |
| P32745 | Somatostatin type 3 (SS3R) [SSTR3] |
| P31391 | Somatostatin type 4 (SS4R) [SSTR4] |
| P35346 | Somatostatin type 5 (SS5R) [SSTR5] |

Tachykinin receptors

| | |
|---|---|
| P25103 | Substance-P (SPR) (NK-1R) [TACR1; TAC1R; NK1R] |
| P21452 | Substance-K (SKR) (neurokinin A) (NK-2R) [TACR2; TAC2R; NK2R] |
| P29371 | Neuromedin K (NKR) (neurokinin B) (NK-3R) [TACR3; TAC3R; NK3R] |
| P30098 | Neuromedin K (NKR) (neurokinin B) (NK-4R) [TACR4] |

Proteinase-activated receptors

| | |
|---|---|
| P25116 | Thrombin [F2R; PAR1; TR] |
| P55085 | Proteinase activated receptor 2 (PAR-2) [PAR2; GPR11] |
| O00254 | Proteinase activated receptor 3 (PAR-3) [PAR3] |

Vasopressin/oxytocin receptors

| | |
|---|---|
| P37288 | Vasopressin V1A (vascular/hepatic-type arginine vasopressin) (AVPR V1A) |
| | [AVPR1A; AVPR1] |
| P47901 | Vasopressin V1B (AVPR V1B) (vasopressin V3) (AVPR V3) [AVPR1B; AVPR3] |
| P30518 | Vasopressin V2 (renal-type arginine vasopressin) (AVPR V2) [AVPR2; ADHR; |
| | V2R] |
| P30559 | Oxytocin (OTR) [OXTR] |

Other receptors

| | |
|---|---|
| P47211 | Galanin type 1 (GAL1-R) (GALR1) [GALNR1; GALNR] |
| 043603 | Galanin type 2 (GAL2-R) (GALR2) [GALNR2; GALR2] |
| O60755 | Galanin type 3 (GAL3-R) (GALR3) [GALNR3; GALR3] |

Orphan receptors

| | |
|---|---|
| P32302 | Burkitt's lymphoma receptor 1 (MONOCYTE-DERIVED RECEPTOR 15) (MDR15) [BLR1] |
| P04201 | MAS proto-oncogene [MAS1; MAS] |
| P35410 | MAS-related MRG [MRG] |
| P35414 | Probable G protein-coupled receptor APJ [AGTRL1; APJ] |
| O75748 | Probable G protein-coupled receptor CHEMR23 [CHEMR23] |
| Q99788 | Chemokine receptor-like 1 (G PROTEIN-COUPLED RECEPTOR DEZ) [CMKRL1; DEZ] |
| Q99527 | Chemokine receptor-like 2 (IL8-related receptor DRY12) [CMKRL2; DRY12; |
| | CEPR; GPR30] |
| P32249 | EBV-induced G protein-coupled receptor 2 (EBI2) [EBI2] |
| P21453 | Probable G protein-coupled receptor EDG-1 [EDG1] |
| Q92633 | Lysophosphatidic acid receptor (EDG-2) [EDG2] |
| Q99500 | Lysosphingolipid receptor (EDG-3) [EDG3] |
| P51686 | Probable G protein-coupled receptor GPR-9-6 |
| P49019 | Probable G protein-coupled receptor HM74 |
| Q15391 | Probable G protein-coupled receptor KIAA0001 [KIAA0001] |
| P25106 | G protein-coupled receptor RDC1 homolog [RDC1] |
| P46091 | Probable G protein-coupled receptor GPR1 [GPR1] |
| P46092 | Probable G protein-coupled receptor GPR2 [GPR2] |
| P46089 | Probable G protein-coupled receptor GPR3 [GPR3] |
| P46093 | Probable G protein-coupled receptor GPR4 (GPR19) [GPR4; GPR19] |
| P46094 | Probable G protein-coupled receptor GPR5 [GPR5] |
| P46095 | Probable G protein-coupled receptor GPR6 [GPR6] |
| P48145 | Probable G protein-coupled receptor GPR7 [GPR7] |
| P48146 | Probable G protein-coupled receptor GPR8 [GPR8] |
| P49683 | Probable G protein-coupled receptor GPR10 [GPR10] |
| P47775 | Probable G protein-coupled receptor GPR12 [GPR12] |
| P49238 | Probable G protein-coupled receptor GPR13 (V28) (CMK-BRL-1) [GPR13] |
| P49685 | Probable G protein-coupled receptor GPR15 [GPR15] |
| Q13304 | Probable G protein-coupled receptor GPR17 [GPR17] |
| Q14330 | Probable G protein-coupled receptor GPR18 [GPR18; GPCRW] |
| Q15760 | Probable G protein-coupled receptor GPR19 (GPR-NGA) [GPR19] |
| Q99678 | Probable G protein-coupled receptor GPR20 [GPR20] |
| Q99679 | Probable G protein-coupled receptor GPR21 [GPR21] |
| Q99680 | Probable G protein-coupled receptor GPR22 [GPR22] |
| Q99705 | Probable G protein-coupled receptor GPR24 (SLC-1) [GPR24; SLC1] |
| O00015 | Probable G protein-coupled receptor GPR25 [GPR25] |
| O00270 | Probable G protein-coupled receptor GPR31 [GPR31] |
| O75388 | Probable G protein-coupled receptor GPR32 [GPR32] |
| O43193 | Probable G protein-coupled receptor GPR38 [GPR38] |
| O43194 | Probable G protein-coupled receptor GPR39 [GPR39] |
| O14842 | Probable G protein-coupled receptor GPR40 [GPR40] |
| O14843 | Probable G protein-coupled receptor GPR41 [GPR41] |
| O15529 | Probable G protein-coupled receptor GPR42 [GPR42] |
| O15552 | Probable G protein-coupled receptor GPR43 [GPR43] |
| Q15743 | Probable G protein-coupled receptor GPT68 [GPR68; OGR1] |

Family 2 (B) receptors

| | |
|---|---|
| P30988 | Calcitonin (CT-R) [CALCR] |
| Q16602 | Calcitonin gene-related peptide type 1 (CGRP TYPE 1) [CGRPR] |
| P34998 | Corticotropin releasing factor 1 (CRF1) [CRHR1; CRHR; CRFR] |
| Q13324 | Corticotropin releasing factor 2 (CRF2) [CRHR2; CRF2R] |
| P48546 | Gastric inhibitory peptide (GIP-R) [GIPR] |
| P47871 | Glucagon (GL-R) [GCGR] |
| P43220 | Glucagon-like peptide 1 (GLP-1-R) [GLP1R] |
| Q02643 | Growth hormone-releasing hormone (GRFR) [GHRHR] |
| Q03431 | Parathyroid hormone/parathyroid hormone-related peptide [PTHR1; PTHR] |
| P49190 | Parathyroid hormone (PTH2) [PTHR2] |
| P41586 | Pituitary adenylate cyclase activating polypeptide type I [ADCYAP1R1] |
| P47872 | Secretin (SCT-R) [SCTR] |
| P32241 | Vasoactive intestinal polypeptide 1 (VIP-R-1) [VIPR1] |
| P41587 | Vasoactive intestinal polypeptide 2 (VIP-R-2) (PACAP-R-3) [VIPR2] |
| P48960 | Leucocyte antigen CD97 [CD97] |
| Q14246 | Cell surface glycoprotein EMR1 [EMR1] |

Family 3 (C) receptors (metabotropic glutamate and calcium receptors)

| | |
|---|---|
| Q13255 | Metabotropic glutamate 1 [GRM1; MGLUR1] |
| Q14416 | Metabotropic glutamate 2 [GRM2; MGLUR2] |
| Q14832 | Metabotropic glutamate 3 [GRM3; MGLUR3] |
| Q14833 | Metabotropic glutamate 4 [GRM4; MGLUR4] |
| P41594 | Metabotropic glutamate 5 [GRM5; MGLUR5] |
| O15303 | Metabotropic glutamate 6 [GRM6; MGLUR6] |
| Q14831 | Metabotropic glutamate 7 [GRM7; MGLUR7] |
| O00222 | Metabotropic glutamate 8 [GRM8; MGLUR8] |
| P41180 | Extracellular calcium-sensing receptor [CASR; PCAR1] |

Once the amino acid sequence of the target GPCR is known, various methods of identifying putative transmembrane domains known in the art can be employed to determine the location of the juxtamembrane extracellular regions. Such methods include, for example, the use of hydropathy plots such as those of Kyte-Doolittle, Hopp-Wood and Eisenberg, or prediction of the location of transmembrane regions by computer modeling using the structure of a known receptor, such as rhodopsin, as a basis. In addition, various publicly available databases, including those listed above, contain detailed information on the domain organization of the GPCRs. The predicted transmembrane domains for a number of GPCRs have also been reported in the literature.

Some exemplary amino acid sequences corresponding to the juxtamembrane extracellular region of known human GPCRs are provided in Table 2. Suitable peptide antagonists may be prepared that comprise all or part of one of the listed sequences, typically at least 3 consecutive amino acids from one of these sequences.

TABLE 2

Exemplary Juxtamembrane Extracellular Amino
Acid Sequences from Known Human GPCRs

| Amino Acid Sequence | SEQ ID NO |
|---|---|
| Prostaglandin F2-alpha receptor | |
| ILGHRDYK | 1 |
| WEDRFYLL | 2 |
| YQDRFYLL | 3 |
| ILAHRDYK | 4 |
| ILGFRDYK | 5 |
| ILGHKDYK | 6 |
| ILGHRNYK | 7 |
| ILGHQDYK | 8 |
| ILGHRDY | 9 |
| ILGWRDYK | 10 |
| ILGXRDYK | 11 |
| SNVLCSIF | 12 |
| Urotensin Receptor | |
| LAMRLVRRG | 13 |
| VIRATRPAL | 14 |
| Glucagon Receptor | |
| LVIDGLLRT | 15 |
| AAVRCGAV | 16 |
| FVTDEHAQ | 17 |
| R14 Orphan Receptor (AAL86878) | |
| HIICSPLR | 18 |
| IFFDSTECW | 19 |
| Platelet Activating Factor (PAF) Receptor | |
| LVPVSGKEY | 20 |
| FLTYRFES | 21 |
| NGQNQYYV | 22 |
| GAVNCLFK | 23 |
| VTNTSDLV | 24 |
| LEALTWPL | 25 |
| LCLTVQHAD | 26 |
| Endothelin (ETA) Receptor | |
| NIYKFAST | 27 |
| KFVNIPLDIV | 28 |
| FLKCLFVG | 29 |
| HQEGRYEFL | 30 |
| WWDKVDQY | 31 |
| ENYVTKKLV | 32 |
| ENYVTKKL | 33 |
| LLECRENKD | 34 |
| a-1A Adrenergic receptor (AR) | |
| VNISKAILLG | 35 |
| VLG YWAFGRVFC | 36 |
| YWAFGRVFCN IW | 37 |
| FGWRQPAPEDET | 38 |
| ICQINEEPGYVL | 39 |
| FFPDFKPSETVF | 40 |
| FFPDFKPSETVF | 41 |
| 5H1A Serotonin receptor (SR) | |
| GISDVTVSYQVI | 42 |
| VLNKWTLGQVTC | 43 |
| KWTLGQVTCD LF | 44 |
| LGWRTPEDRSDP | 45 |
| CTISKDHGYTIY | 46 |
| VLPFCESSCHMP | 47 |
| FCESSCHMPTLL | 48 |
| Adenosine A1 receptor | |
| PPSISAFQAAYI | 49 |
| LIN IGPQTYFHT | 50 |
| IGPQTYFHTCLM | 51 |
| WNNLS AVERAV | 52 |
| EPVIKCEFEKVI | 53 |
| LFCPSCHKPSIL | 54 |
| LFCPSCHKPSIL | 55 |
| Melanocortin (MC) 4 receptor | |
| GYSDGGCYEQLF | 56 |
| LLNST DTDAQSF | 57 |
| AQSFTVNIDNVI | 58 |
| IIYSDSSAVIIC | 59 |
| LFIIYSDSSAVI | 60 |
| YISCPQNPYCVC | 61 |
| SCPQNPYCVCFM | 62 |
| Protease activated receptor-2 (PAR-2) | |
| ASVLTGKLTTVF | 63 |
| FPLKIAYHIHGN | 64 |
| NWIYGEALCNVL | 65 |
| LYVVKQTIFIPA | 66 |
| PEQLLVGDMFNY | 67 |
| VVHYFLIKSQGQ | 68 |
| FLIKSQGQSHVY | 69 |
| Metabotropic glutamate receptor 1 (MGR1) | |
| RYLEWSNIEPII | 70 |
| LIAKPTTTSCYLQ | 71 |
| PTTTSCYLQRLL | 72 |
| IIMEPPMPILSY | 73 |
| LICNTSNLGVV | 74 |
| YFGSNYKIITTC | 75 |
| FGSNYKIITTCF | 76 |
| Parathyroid hormone (PTH) receptor | |
| FLMEYHMQ | 77 |

In one embodiment of the present invention, the peptide antagonists comprise an amino acid sequence of about 6 to about 9 amino acids corresponding to the sequence of a juxtamembrane extracellular region of a mammalian GPCR. In another embodiment, the peptide antagonists comprise an amino acid sequence of about 7 to about 9 amino acids corresponding to the sequence of a juxtamembrane extracellular region of a mammalian GPCR. In a further embodiment, the peptide antagonists comprise an amino acid sequence of about 8 to about 9 amino acids corresponding to the sequence of a juxtamembrane extracellular region of a mammalian GPCR.

As is known in the art, substitution of all L-amino acids within the peptide with D-amino acids results in either an “inverso” peptide, or in a “retro-inverso” peptide (see Goodman et al. “Perspectives in Peptide Chemistry” pp. 283-294 (1981); U.S. Pat. No. 4,522,752). An “inverso” peptide is one in which all L-amino acids of a sequence have been replaced with D-amino acids, and a “retro-inverso” peptide is one in which the sequence of the amino acids has been reversed (“retro”) and all L-amino acids have been replaced with D-amino acids. For example, if the parent peptide is Thr-Ala-Tyr, the retro form is Tyr-Ala-Thr, the inverso form is thr-ala-tyr, and the retro-inverso form is tyr-ala-thr (lower case letters indicating D-amino acids). Compared to the parent peptide, a retro-inverso peptide has a reversed backbone while retaining substantially the original spatial conformation of the side chains, resulting in an isomer with a topology that closely resembles the parent peptide. The present invention contemplates GPCR peptide antagonists that are retro peptides or retro-inverso peptides.

In accordance with one embodiment of the present invention, the peptide antagonists comprise all D-amino acids. In a further embodiment, the peptide antagonists are D-peptides that optionally comprise one or more L-amino acids.

In an alternate embodiment of the present invention, the peptide antagonists comprise at least one amino acid that has a D-configuration. Peptide antagonists comprising at least two, at least three, at least four amino acids, at least five, at least six, and at least seven amino acids having a D-configuration are also contemplated.

In accordance with the present invention, the peptide antagonists may further comprise one or more modifications or additional amino acids, which do not correspond to the sequence of the GPCR, at the N-terminus, the C-terminus, or both the N- and C-termini. The presence of extra amino acids or modifications to one of the termini of the peptides may be desirable, for example, to improve the stability of the peptides, to incorporate a “tag” to aid in identification, detection or purification protocols, to improve solubility or to improve pharmacokinetic parameters. By way of example, the solubility of the peptides may be improved by the addition of certain amino acids at the C-terminus. Examples of suitable amino acids that can be added at the C-terminal end to improve the solubility of the peptides include, but are not limited to, Gly-Lys and Gly-Lys-Lys. Non-limiting examples of suitable modifications that may be made at the N-terminus include the addition of a R—CO— or a R—O—CO— group, wherein R is an alkyl, heteroalkyl, a heterocyclic ring, a heteroaromatic ring or an aromatic ring. Non-limiting examples of suitable R—CO— groups are benzoyl, acetyl, tert-butyl acetyl, para-phenyl benzoyl, trifluoroacetyl, cyclohexylcarbonyl and phenylacetyl.

In one embodiment of the present invention the peptide antagonist comprises general formula I:

X-A-B-Y (I)

wherein

X is either absent or independently selected from the group comprising: one to four amino acids, a R—CO— or a R—O—CO— group, wherein R is an alkyl, heteroalkyl, a heterocyclic ring, a heteroaromatic ring or an aromatic ring;

A is between 2 and 3 hydrophobic or neutral amino acids;

B is between 4 and 8 hydrophilic or neutral amino acids; and

Y is either absent or independently selected from a group comprising: one to four amino acids, an hydroxyl group, Gly-Lys and Gly-Lys-Lys, and wherein the sequence A-B corresponds to the sequence of a juxtamembrane extracellular region of a GPCR.

In the context of the present invention, a “hydrophobic” amino acid refers to an amino acid selected from the group of: alanine, cysteine, isoleucine, leucine, methionine, phenylalanine, proline, tryptophan, tyrosine and valine. A “hydrophilic” amino acid refers to an amino acid selected from the group of: arginine, asparagine, aspartate, glutamine, glutamate, histidine, lysine, serine and threonine. In general, glycine is considered to be a “neutral” amino acid.

In another embodiment of the present invention, the peptide antagonist comprises an amino acid sequence of about 5 and about 10 amino acids corresponding to a juxtamembrane extracellular region of a GPCR, the amino acid sequence comprising at least 3 hydrophobic amino acids.

In another embodiment of the present invention, the peptide antagonists comprise a sequence of three or more consecutive amino acids of a sequence selected from the group comprising: SEQ ID NOs:1 to 20, 29, 31, 33 and 77, or a peptide analogue, derivative, variant or peptidomimetic thereof. In another embodiment, the peptide antagonists comprise a sequence of three or more consecutive amino acids of any one of SEQ ID NOs:13 to 20, 29, 31, 33 or 77, or a peptide analogue, derivative, variant or peptidomimetic thereof.

In still another embodiment, the peptide antagonists comprise a sequence of three or more consecutive amino acids of a sequence selected from the group comprising: lamrlvrrG (SEQ ID NO:78); viratrpal (SEQ ID NO:79); IvidGllrt (SEQ ID NO:80); aavrcGav (SEQ ID NO:81); fvtdehaq (SEQ ID NO:82); hiicsplr (SEQ ID NO:83); iffdstecw (SEQ ID NO:84); lvpvsGkey (SEQ ID NO:85); flmeyhmq (SEQ ID NO:86); flkclfvG (SEQ ID NO:87); wwdkvdqy (SEQ ID NO:88); enyvtkkl (SEQ ID NO:89); yqdrfyll (SEQ ID NO:90); ilghrdyk (SEQ ID NO:91); ilahrdyk (SEQ ID NO:92); ilAhrdyk (SEQ ID NO:93); ilgfrdyk (SEQ ID NO:94); ilghkdyk (SEQ ID NO:95); ilghknyk (SEQ ID NO:96); ilghqdyk (SEQ ID NO:97); ilghrdy-amide (SEQ ID NO:98); ilghrdyk-amide (SEQ ID NO:99); ilgwrdyk (SEQ ID NO:100); ilgxrdyk (SEQ ID NO:101) and snvlcsif (SEQ ID NO:102).

In another embodiment, the peptide antagonists comprise a sequence of three or more consecutive amino acids of any one of SEQ ID NOs:78 to 89.

In still another embodiment, the peptide antagonists comprise a sequence of three or more consecutive amino acids of any one of the sequences set forth in Table 3.

TABLE 3
Exemplary Peptide Antagonist Sequences

| Amino Acid Sequence | SEQ ID NO |
|---|---|
| ilghrdyk | 91 |
| wedrfyll | 103 |
| yqdrfyll | 90 |
| ilahrdyk | 92 |
| ilgfrdyk | 94 |
| ilghkdyk | 95 |
| ilghrnyk | 104 |
| ilghqdyk | 97 |
| ilghrdy | 105 |
| ilgwrdyk | 100 |
| ilgxrdyk | 101 |
| snvlcsif | 102 |
| lamrlvrrg | 106 |
| viratrpal | 79 |
| lvidgllrt | 107 |
| aavrcgav | 108 |
| fvtdehaq | 82 |
| hiicsplr | 83 |
| iffdstecw | 84 |
| lvpvsgkey | 109 |
| fltyrfes | 110 |
| ngqnqyyv | 111 |
| gavnclfk | 112 |
| vtntsdlv | 113 |
| lealtwpl | 114 |
| lcltvqhad | 115 |
| niykfast | 116 |
| kfvnipldiv | 117 |
| flkclfvg | 118 |
| hqegryefl | 119 |
| wwdkvdqy | 88 |
| enyvtkklv | 89 |
| enyvtkkl | 120 |
| llecrenkd | 121 |
| vniskaillg | 122 |
| vlg ywafgrvfc | 123 |
| ywafgrvfcn iw | 124 |
| fgwrqpapedet | 125 |
| icqineepgyvl | 126 |
| ffpdfkpsetvf | 127 |
| ffpdfkpsetvf | 128 |
| gisdvtvsyqvi | 129 |
| vlnkwtlgqvtc | 130 |
| kwtlgqvtcdlf | 131 |
| lgwrtpedrsdp | 132 |
| ctiskdhgytiy | 133 |
| vlpfcesschmp | 134 |
| fcesschmptll | 135 |
| ppsisafqaayi | 136 |
| linigpqtyfht | 137 |
| igpqtyfhtclm | 138 |
| wnnlsaverav | 139 |
| epvikcefekvi | 140 |
| lfcpschkpsil | 141 |
| lfcpschkpsil | 142 |
| gysdggcyeqlf | 143 |
| llnst dtdaqsf | 144 |
| aqsftvnidnvi | 145 |
| iiysdssaviic | 146 |
| lfiiysdssavi | 147 |
| yiscpqnpycvc | 148 |
| scpqnpycvcfm | 149 |
| asvltgklttvf | 150 |
| fplkiayhihgn | 151 |
| nwiygealcnvl | 152 |
| lyvvkqtifipa | 153 |
| peqllvgdmfny | 154 |
| vvhyfliksqgq | 155 |
| fliksqgqshvy | 156 |
| rylewsniepii | 157 |
| liakptttscylq | 158 |
| ptttscylqrll | 159 |
| iimeppmpilsy | 160 |
| licntsnlgvv | 161 |
| yfgsnykiittc | 162 |
| fgsnykiittcf | 163 |
| flmeyhmq | 86 |

In other embodiments, the peptide antagonists comprise a sequence of four or more, five or more, and six or more, consecutive amino acids from any one of SEQ ID NOs:1 to 163.

In another embodiment, the peptide antagonists comprise one of SEQ ID NOs:1 to 20, 29, 31, 33, 77 to 102, or a peptide analogue, derivative, variant or peptidomimetic thereof, and one or more additional amino acids at the C-terminus that do not correspond to the sequence of the glucagon receptor. Examples of such additional amino acids include Gly-Lys and Gly-Lys-Lys.

In another embodiment, the peptide antagonists comprise one of SEQ ID NOs:1 to 20, 29, 31, 33, 77 to 102, or a peptide analogue, derivative, variant or peptidomimetic thereof, and one or more modifications at the N-terminus. Examples of such modifications include the addition of a R—CO— or a R—O—CO— group to the N-terminus of the peptide.

### Preparation of the Candidate Peptide Antagonists

The candidate peptide antagonists can be readily prepared by standard chemical synthesis techniques. The principles of solid phase chemical synthesis of polypeptides are well known in the art and may be found in general texts in the area such as Dugas, H. and Penney, C., Bioorganic Chemistry (1981) Springer-Verlag, New York, pgs. 54-92; Merrifield, J. M., Chem. Soc., 85:2149 (1962), and Stewart and Young, Solid Phase Peptide Synthesis, pp. 24-66, Freeman (San Francisco, 1969). Methods of preparing peptide analogues, derivatives and peptideomimetics are well known in the art.

Covalent modifications of the peptide can be introduced, for example, by reacting targeted amino acid residues with an organic derivatizing agent that is capable of reacting with selected side chains or terminal residues as is known in the art.

For example, cysteinyl residues may be reacted with α-haloacetates (and corresponding amines), such as 2-chloroacetic acid or chloroacetamide, to give carboxymethyl or carboxyamidomethyl derivatives. Cysteinyl residues may also be derivatized by reaction with compounds such as bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues may be derivatized by reaction with compounds such as diethylprocarbonate, which is relatively specific for the histidyl side chain, e.g. by reaction with this reagent at pH 5.5-7.0. Para-bromophenacyl bromide may also be used, for example, by reaction in 0.1M sodium cacodylate at pH 6.0.

Lysinyl and amino terminal residues may be reacted with compounds such as succinic or other carboxylic acid anhydrides. Derivatization with these agents is expected to have the effect of reversing the charge of the lysinyl residues. Other suitable reagents for derivatizing α-amino-containing residues include compounds such as imidoesters, for example, methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with one of several conventional reagents, including, but not limited to, phenylglyoxal; 2,3-butanedione; 1,2-cyclohexanedione and ninhydrin according to known methods. Derivatization of arginine residues typically requires that the reaction be performed in alkaline conditions due to the high pKa of the guanidine functional group. One skilled in the art will understand that these reagents may react with the groups of lysine as well as the arginine epsilon-amino group.

Methods of modifying tyrosyl residues are well-known. For example, introduction of spectral labels into tyrosyl residues by reaction with aromatic diazonium compounds or tetranitromethane. N-acetylimidizol and tetranitromethane may be used to form O-acetyl tyrosyl species and 3-nitro derivatives, respectively.

Carboxyl side groups (aspartyl or glutamyl) may be selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Aspartyl and glutamyl residues can also be converted to asparaginyl and glutaminyl residues by reaction with ammonium ions.

Glutaminyl and asparaginyl residues may be deamidated to the corresponding glutamyl and aspartyl residues, for example, through the use of mildly acidic conditions. Either form of these residues falls within the scope of the present invention.

Derivatization with bifunctional agents is useful for cross-linking the peptide antagonist to a water-insoluble support matrix or to other macromolecular carriers, according to known method steps. Commonly used cross-linking agents include, but are not limited to, 1,1-bis(diazoacetyl)-2-phenylethane, glutaraldehyde, N-hydroxysuccinimide esters (such as esters with 4-azidosalicylic acid), homobifunctional imidoesters, including disuccinimidyl esters such as 3,3′-dithiobis(succinimidylpropionate), and bifunctional maleimides such as bis-N-maleimido-1,8-octane. Derivatizing agents, such as methyl-3-[(p-azidophenyl)dithio]propioimidate, yield photoactivatable intermediates that are capable of forming cross-links in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 (which are herein incorporated entirely by reference), may be employed for protein immobilization.

Other possible modifications of the peptide antagonists include hydroxylation of proline and lysine, phosphorylation of hydroxyl groups of seryl or threonyl residues, methylation of the alpha-amino groups of lysine, arginine, and histidine side chains (see T. E. Creighton, Proteins: Structure and Molecule Properties, W. H. Freeman & Co., San Francisco, pp. 79-86 (1983)), acetylation of the N-terminal amine, methylation of main chain amide residues (or substitution with N-methyl amino acids) and, in some instances, amidation of the C-terminal carboxyl groups, according to known method steps.

The peptides of the present invention may also be prepared in their salt form. The peptides may be sufficiently acidic or sufficiently basic to react with a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like.

Base addition salts include those derived from inorganic bases, such as ammonium or alkali or alkaline earth met al hydroxides, carbonates, bicarbonates, and the like. Such bases useful in preparing the salts of this invention may be selected from the group of sodium hydroxide, potassium hydroxide, ammonium hydroxide, potassium carbonate, and the like.

A worker skilled in the art will readily understand that when the peptides in salt form are for therapeutic purposes, the salt will be a pharmaceutically acceptable salt.

### Efficacy of the Candidate Peptide Antagonists

In accordance with the present invention, a peptide antagonist is capable of selectively modulating the function of the receptor from which it is derived. Candidate peptide antagonists are, therefore, tested for their ability to modulate GPCR function. The GPCR function may be a cellular or physiological event that is directly or indirectly associated with a GPCR in vivo. Thus, the peptide antagonist may affect, for example, GTP binding and/or hydrolysis, cellular calcium levels, phosphoinositide hydrolysis, cellular cAMP levels, adenyl cyclase activation or inhibition, protein kinase A activity, phospholipase C activity, cell growth and/or differentiation, gene expression, smooth muscle contraction or dilation, vasoconstriction or dilation, nerve cell membrane potential, secretion from glandular cells, and the like. Methods of testing the ability of the candidate peptide antagonists to modulate GPCR function can be conducted in vitro, ex vivo or in vivo.

An exemplary method of testing the ability of the candidate peptide antagonists in vitro or ex vivo comprises the steps of:

1. culturing cells in which the target GPCR is expressed;

2. contacting the cells with the candidate peptide antagonist; and

3. measuring at least one cellular and/or physiological consequence of modulation of GPCR function.

The cells employed in the above method may express the GPCR naturally or they may be genetically engineered to express the GPCR by standard gene transfer technologies. As an alternative to cultured cells, mammalian tissue in which the target GPCR is known to exist may be employed in an ex vivo method. The assay may also include a step in which the cultured cells or tissue sample are contacted with a suitable amount of an agonist of the target GPCR. One skilled in the art will understand that the sequence in which the cells/tissue are contacted with the agonist and candidate peptide antagonist may be varied in order to study different effects of the antagonist. For example, by contacting the cells/tissue with the candidate peptide antagonist prior to the agonist the ability of the candidate peptide antagonist to block or prevent the effect(s) of the agonist on the GPCR can be assessed. In contrast, the ability of the candidate peptide antagonist to inhibit or reduce the effect(s) brought about by the binding of the agonist to the GPCR can be assessed by contacting the cells/tissue first with the agonist and then, after a suitable period of time, with the candidate peptide antagonist.

Changes in cellular and/or physiological effects mediated by GPCRs can be measured using a number of standard techniques known in the art. Exemplary methods are provided herein in the Examples. One skilled in the art will understand that other standard methods may also be employed.

Assays may be conducted to assess the ability of the candidate peptide antagonists to modulate GPCR function in vivo. As indicated above with respect to in vitro and ex vivo assays, the ability of the candidate peptide antagonist to modulate the function of a target GPCR can be measured in the presence or absence of an agonist and, in the former case, the sequence for administration of the candidate peptide and agonist to the test animal may be varied. Such assays are familiar to those versed in the art and are described in detail in numerous scientific publications and methods manuals.

Selection of an appropriate test animal will be dependant on the target GPCR being studied. Examples of suitable animals include mice, rats, guinea pigs, rabbits, piglets and pigs. Various physiological effects associated with a target GPCR that can be monitored in vivo or ex vivo in order to assess the efficacy of a candidate peptide are known in the art. Representative non-limiting examples are provided in Table 4.

TABLE 4
Measurable Physiological Effects Associated with Various GPCRs

| GPCR | Physiological effect | | Assay/Test Animal | |
|---|---|---|---|---|
| Endothelin receptor | | Endothelin induced vascular | | Porcine retinal microvascular |
| | | constriction | | contraction assay¹ |
| Prostaglandin F_{2α} | 1. | PGF_{2α}induced vascular | 1. | Porcine retinal microvascular |
| (PGF_{2α}) receptor | | constriction | | contraction assay¹ |
| | 2. | Muscle contraction | 2. | Uterine smooth muscle |
| | | | | contraction assay³ |
| Prostaglandin EP₄ | 1. | Urinary flow and glomerular | 1. | Rat model² |
| receptor | | filtration rates | | |
| | 2. | PGE2 induced vasodilation | 2. | Sheep fetal ductus arteriosus |
| | | | | or piglet saphenous vein² |
| Glucagon receptor | | Glucagon induced hyperglycemia | | Rat model³ |
| R14 orphan receptor | | Intraocular pressure | | Piglet model³ |
| PAF receptor | 1. | C-PAF induced hypotension | 1. | Piglet model³ |
| | 2. | C-PAF induced vascular | 2. | Porcine retinal microvascular |
| | | constriction | | contraction assay¹ |
| Urotensin II receptor | 1. | Urotensin II induced hypertension | 1. | Piglet model³ |
| | 2. | Urotensin II induced bronchial | 2. | Bronchial ring assay⁴ |
| | | constriction | | |
| | 3. | Urotensin II induced vascular | 3. | Porcine retinal microvascular |
| | | constriction | | contraction assay¹ |

¹See, for example, Li et al, (1996) J. Pharmacol. Exp. Ther. 278: 370-377.
²See, for example, U.S. Patent Application 2003/0017988.
³See, for example, the methods provided herein as Examples IV, V, VIII, X, XVI and XVII.
⁴See for example, Hay et al, (2000) Br. J. Pharmacol. 131: 10-12 and Example IX.

Antagonist peptides found to be efficacious in the above assays can be further characterized if required, for example, by testing varying concentrations of the peptide antagonist against a fixed concentration of agonist to determine the potency of the antagonist compound. Varying the concentration of the agonist while maintaining a fixed peptide antagonist concentration will provide information as to whether the action of the antagonist is competitive or non-competitive and measuring the effect of the peptide antagonist on one or more distantly-related GPCR can provide information about the selectivity of the antagonist.

### Pharmaceutical Compositions

For in vivo use, the peptide antagonists may be formulated as pharmaceutical compositions with an appropriate pharmaceutically physiologically acceptable carrier, diluent, excipient or vehicle. The pharmaceutical compositions comprise one or more peptide antgonist and may further optionally comprise one or more other pharmaceutical compounds.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

The pharmaceutical compositions can be formulated in unit dosage form. The term “unit dosage form” refers to a physically discrete unit suitable as a unitary dosage for a mammal, such as a human, each unit containing a predetermined quantity of peptide calculated to produce the desired therapeutic effect in association with a suitable pharmaceutical excipient. For example, a suitable unit dosage form for the peptides of the invention may be one containing a dosage from about 10 μg to about 10 mg of each peptide.

The present invention also contemplates controlled release preparations. Such preparations usually comprise one or more polymer that serves to complex or absorb the peptide antagonist. Examples of such polymers include, but are not limited to, polyesters, polyamino acids, polyvinylpyrrolidone, ethylenevinyl acetate, methylcellulose, carboxymethylcellulose, and protamine sulfate, in an appropriate concentration and according to various methods of incorporation.

The duration of action of the peptide antagonist may also be controlled by incorporating the peptide into particles of a polymeric material. For example, particles comprising polyesters, polyamino acids, hydrogels, poly (lactic acid) or ethylene vinylacetate copolymers.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philidelphia, Pa. (2000) (formerly “Remingtons Pharmaceutical Sciences”).

### Uses

In accordance with the present invention, the peptide antagonists are capable of modulating the function of a target GPCR. The present invention, therefore, provides for methods of modulating GPCR function in a mammal comprising administering an effective amount of one or more GPCR peptide antagonists. The peptide antagonists can be used to modulate a cellular or physiological effect mediated by the target GPCR in vivo. The peptide antagonists can also be used in the treatment and/or prevention of GPCR-mediated diseases, disorders and conditions. Thus, the present invention also provides methods of preventing, ameliorating or treating diseases or conditions in a mammal that are associated with changes in GPCR activity.

One skilled in the art will appreciate that an “effective amount” of a peptide antagonist will vary depending on the particular peptide antagonist being employed, the mode of administration and the subject being treated, for example, the dosage will vary depending on the seriousness of the disorder and the age and weight of the patient, among other factors. Appropriate dose ranges can be determined by a medical practitioner. An example of a typical daily dosage (such as a single, daily dose) to be administered is one within the range from about 1 μg/kg to about 10 mg/kg of body weight, although lower or higher dosages may be administered according to the judgement of the medical practitioner.

Examples of cellular or physiological effects mediated by GPCRs that may be modulated using one or more of the peptide antagonist of the invention include, but are not limited to, muscle contraction, vasoconstriction or dilation, blood glucose levels, intra-ocular pressure, blood pressure, activation or inhibition of gene transcription and cell proliferation.

Non-limiting examples of diseases, disorders and conditions associated with aberrant GPCR function.that may be treated and/or prevented using one or more of the peptide antagonists of the invention include, endotoxin or septic shock; sepsis; diabetes (Type I and II) and conditions associated therewith; hyperglycemia; cardiovascular diseases, including heart failure, angina pectoris, arrhythmias, myocardial infarction, cardiac hypertrophy, hypotension, hypertension (e.g. essential hypertension); renal hypertension; pulmonary hypertension; thrombosis; cerebral vasospasm; subarachnoid hemorrhage; cerebral ischemia; cerebral infarction; peripheral vascular disease; Raynaud's disease; kidney disease (e.g. renal failure); urinary retention; asthma; allergies; glaucoma and stroke.

Other examples include psychiatric and CNS disorders, including schizophrenia, episodic paroxysmal anxiety (EPA) disorders such as obsessive compulsive disorder (OCD), post traumatic stress disorder (PTSD), phobia and panic, major depressive disorder, bipolar disorder, Parkinson's disease, general anxiety disorder, autism, delirium, multiple sclerosis, Alzheimer disease/dementia and other neurodegenerative diseases, severe mental retardation, dyskinesias, Huntington's disease, Tourett's syndrome, tics, tremor, dystonia, spasms, anorexia, bulimia, arteriosclerosis, addiction/dependency/craving, sleep disorder, epilepsy, migraine, attention deficit/hyperactivity disorder (ADHD), dyslipidemias; obesity; emesis; gastrointestinal disorders, including irritable bowel syndrome (IBS), inflammatory bowel disease (IBD), gastroesophagal reflux disease (GERD), motility disorders and conditions of delayed gastric emptying, such as post operative or diabetic gastroparesis, ulcers (e.g. gastric ulcer); diarrhea; other diseases including osteoporosis; inflammations; infections such as bacterial, fungal, protozoan and viral infections, including infections caused by HIV-1 or HIV-2; pain; cancers; chemotherapy induced injury; tumor invasion; immune disorders; arthritis; benign prostatic hypertrophy; and gynecological disorders.

For treatment of certain diseases, the peptide antagonists of the present invention may be used as part of a therapeutic regimen that includes other drugs. Additionally, the peptide antagonists may also be used as part of a therapeutic regimen that includes diet and/or exercise modification.

One or more of the peptide antagonists may be formulated into a pharmaceutical composition in combination with one or more other pharmaceutical agents for administration to a subject. Alternatively, the one or more peptide antagonist and the pharmaceutical agent(s) may be formulated separately. When separate formulations are used, they may be administered to the subject concurrently or they may be administered at different times.

To gain a better understanding of the invention described herein, the following examples are set forth. It should be understood that these examples are for illustrative purposes only. Therefore, they should not limit the scope of this invention in any way.

### EXAMPLES

### Preparation of Inhibitors to the Prostaglandin F_{2α} (FP) Receptor: Chemical Synthesis of PCP-8 and PCP-10

The peptide antagonists PCP-8 and PCP-10, which are 8 amino acids in length, were synthesized using F-moc chemistry and solid phase Merrifield method. These peptides were purified by HPLC and their purity tested by mass spectroscopy.

The novel strategy of using peptides derived from the extracellular domains of the FP receptor to inhibit the signal transduction and the functional consequences of FP receptor can be generalized to all G protein-coupled receptors. Peptides derived from the first and second extracellular loops of FP receptor were found to be effective inhibitors of FP receptor as illustrated below.

### Example I

### Effects of Peptides, PCP-8 and PCP-10, on Ligand-Induced Phosphoinositide Hydrolysis in Mammalian Cells Overexpressing the FP Receptor

Both PCP-8 and -10 were tested in HEK293 cells expressing the human FP receptor. For this purpose, HEK 293 cells stably expressing human FP receptor were plated in 12-well plates in DMEM medium containing 10% fetal bovine serum, penicillin (10 U/ml) and streptomycin (10 μg/ml) and cultured in a humidified atmosphere containing 5% CO₂ at 37° C. After the wells were 80% confluent, the cells were labeled with 2 μCi/ml of [³H]-myo inositol overnight. Next day, the cells were washed once with PBS, and incubated in 0.5 ml of Kreb's buffer containing 10 mM LiCl and indicated concentrations of PCP peptides for 30 min. PGF₂a, at 1 μM was added to the cells and the incubation was carried out for an additional 30 min. The cells were solubilized with 0.1 N NaOH for 10 min and neutralized with 0.1 N formic acid. The lysates were collected and 1 ml each of methanol and chloroform were sequentially added and vortexed briefly. After centrifugation to separate the phases, inositol phosphates were separated by ion exchange chromatography as described below (Berridge,M. J.et 30 al., 1983, Biochem. J. 212:473-482).

Briefly, the medium was discarded and the IP3 synthesis was stopped by adding 0.6 ml ice-cold methanol. The cells were scraped and collected into polypropylene tubes. Distilled water (0.5 ml) and chloroform (0.6 ml) were added and vigorously vortexed for 2 min. The phases were separated by centrifugation at 6000×g for 10 min. The aqueous phase was applied to AG-1X-8TM (Formate form) anion exchange columns (1 ml bed volume) and free inositol was eluted with 10 ml of water, followed by 60 mM ammonium formate in 0.1 M formic acid. Then, the inositol phosphates were eluted with 5 ml of 1.2 M ammonium formate in 0.1 M formic acid. After adding 3 volumes of scintillation cocktail (Optiphase-HiSafe III), the eluates were counted by scintillation spectrophotometry.

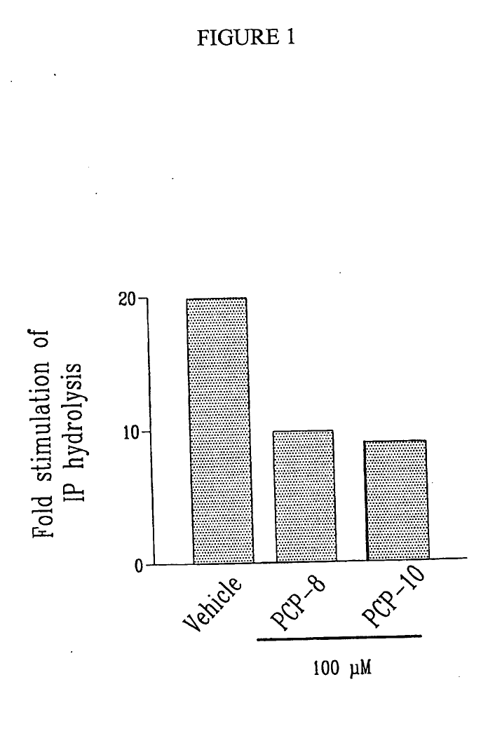

The results of these experiments are shown in FIG. 1. Data are expressed as fold stimulation of inositol phosphate synthesis by 1 μM PGF_{2α} compared to the unstimulated controls. Both PCP-8 and -10 at 100 AM potently inhibited inositol phosphate synthesis initiated by the action of PGF₂ₐ, on FP receptor. The half maximal inhibitory concentrations for both PCP-8 and -10 were slightly less than 100 μM.

### Example II

### Testing PCP Peptides in Porcine Eyecup Model of ex vivo Vasomotricity Assay

In order to see if the peptides could inhibit FP function using an ex vivo model, we chose porcine eyecup model, an ex vivo assay of vascular constriction in porcine retinas which we previously described and validated (Li et al., 1996 J. Pharmacol. Expt. Therapeut. 278: 370-377; Li et al., 1997 Am. J. Physiol. 273:R1283-90; Abran et al., 1997 Am. J. Physiol. 272: R9951001). Since FP receptor densities in newborn vasculature are minimal due to down regulation by high levels of circulating prostaglandins in the perinatal period, the newborn pigs were treated with a prostaglandin synthetase blocker, ibuprofen (30 mg/Kg of bodyweight/8 h for 24 h) to increase the density of the receptors as well as their vasomotor effects. By inhibiting circulating prostaglandins, we were able to show potent inhibition of FP receptor-mediated second messenger synthesis as well as FP-mediated vascular constriction in this eyecup model.

To prepare eyecups, a circular incision was made 3-4 mm posterior to ora serrata to remove the interior segment and vitreous body with minimal handling of the retina. The remaining eyecup was fixed with pins to a wax base in a 20 ml tissue bath containing 20 ml of Kreb's buffer (pH 7.35-7.45), protease inhibitors, leupetin and aprotinin (10 pg/ml each), and equilibrated with 21% oxygen and 5% carbon dioxide at 37° C. The preparations were allowed to stabilize for 30 min. Peptides at 100 μM were added and incubation was continued for 30 min before the addition of PGF_{2α}.

Cumulative concentration-responses of PGF_{2α} and TxA₂ mimetic, U46619, (10^{-1°} to 10⁻⁵ M) curves were constructed separately. To assess the reversibility of the antagonists, the eyecups were thoroughly washed (which would wash away the peptide) with incubation medium and concentration response curves for PGF_{2α} were determined. The outer vessel diameter was recorded with a video camera mounted on a dissecting microscope (Zeiss M 400TM) and the responses were quantified by a digital image analyzer (Sigma Scan Software, Jandel Scientific, Corte Madera, Calif.). Vascular diameter was recorded before and 10 min following the topical application of the agent. Each measurement was repeated three times and showed <1% variability.

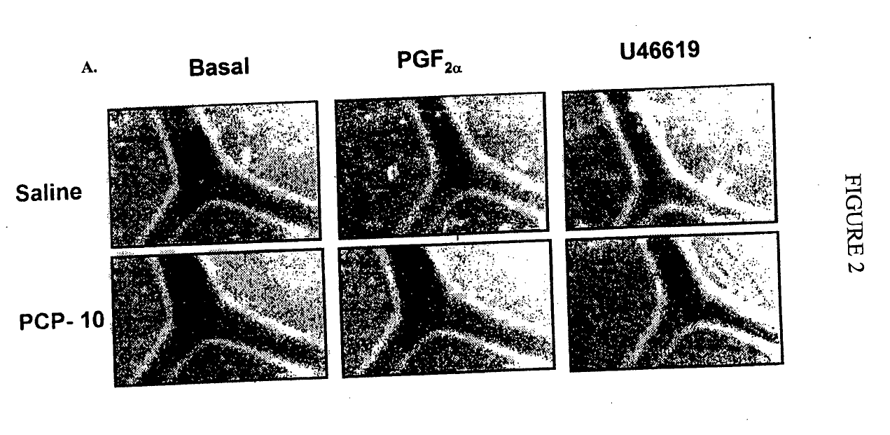

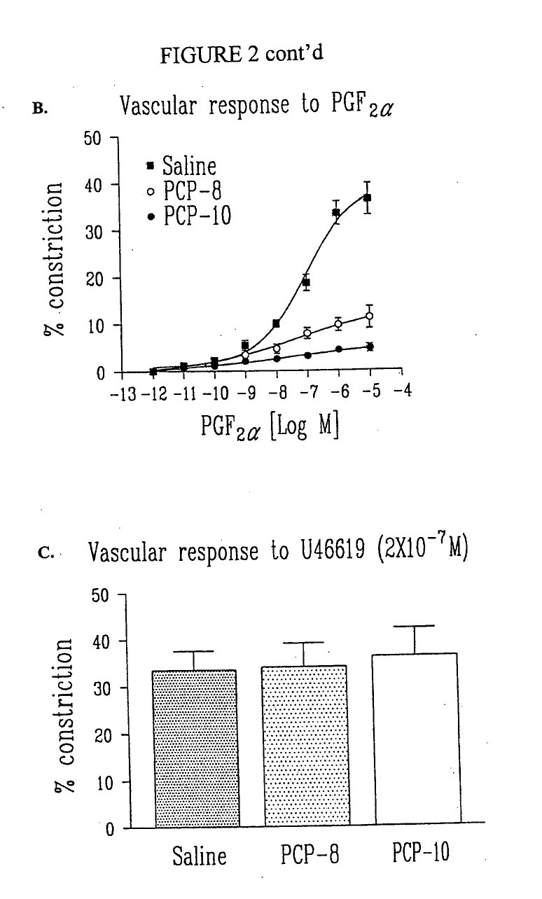

The results are shown in FIG. 2. The peptide PCP-10 had no effect on the basal tone (diameter of the microvessel) of the vessel (FIG. 2A; left panels). Addition of 1 μM of PGF_{2α} potently constricted the vessel in the absence of the peptide (middle-top panel), whereas presence of PCP-10 at 100 μM markedly inhibited PGF_{2α}-mediated vasoconstriction (middle-bottom panel).

The peptide had no effect on the vasoconstriction effected by 1 IM TxA₂ mimetic, U46619, (right panels) acting on another prostanoid receptor coupled to constriction, namely TP receptor. Similar results were obtained for PCP-8 as well. A dose response of PGF_{2α} on the vascular diameter in the presence/absence of PCP-8 and PCP-10 peptides are presented in FIG. 2B. Both peptides abrogated the vasomotor responses even at concentrations exceeding 1 μM of PGF_{2α} suggesting, as expected, that the peptides may be acting in a non-competitive fashion. However, the peptides had no effect on vasoconstriction produced by thromboxane A₂ (FIG. 2C).

Similarly, a peptide derived from the first extracellular loop of FP receptor, PCP-15, inhibited PGF_{2α}-induced constriction (10⁻⁷ M) (88.10 over untreated control; Table 4).

### Example III

### Testing Peptide Variants of PCP-8 in Porcine Eyecup Model of ex vivo Vasomotricity Assay

In order to understand the structural requirements of PCP-8 in its inhibitory action on PGF_{2α}-induced vasoconstriction, different amino acids in PCP-8 sequence were replaced with other D- or L- amino acids and the resulting peptides were chemically synthesized and tested in porcine eyecup model of ex vivo vasomotricity assay. These peptide variants are listed in Table 5.

TABLE 5

Amino acid sequences of peptide
variants of PCP-8 and their inhibitory
potency in porcine eyecup model
of ex vivo vasomotricity assay

| | | | | |
|---|---|---|---|---|
| | % Vasomotor | | | |
| | Response (of | 5 Inhibition | | |
| | maximum | of Maximal | Peptide | |
| Peptide PCP- | constriction)¹ | Response² | Sequence | SEQ ID NO: |

| | | | | |
|---|---|---|---|---|
| 8 | 50.0 | 50.0 | ILGHRDYK | 1 |
| 10 | 20.0 | 80.0 | WEDRFYLL | 2 |
| 14 | 36.0 | 64.0 | yqdrfyll | 90 |
| 13 | 20.0 | 80.0 | ilghrdyk | 91 |
| 13.7 | 23.8 | 76.2 | ilahrdyk | 92 |
| 13.8 | 46.8 | 53.2 | ilAhrdyk | 93 |
| 13.11 | 13.0 | 87.0 | ilgfrdyk | 94 |
| 13.13 | 36.9 | 63.1 | ilghkdyk | 95 |
| 13.14 | 40.3 | 59.7 | ilghknyk | 96 |
| 13.18 | 30.0 | 70.0 | ilghqdyk | 97 |
| 13.20 | 49.6 | 50.4 | ilghrdy-amide | 98 |
| 13.21 | 46.2 | 53.8 | ilghrdyk-amide | 99 |
| 13.22 | 16.6 | 83.4 | ilgwrdyk | 100 |
| 13.24 | 6.2 | 93.8 | ilgxrdyk | 101 |
| 15 | 11.9 | 88.1 | snvlcsif | 102 |

¹Percent vasomotor response in the presence of 100 μM peptide is calculated as percent change in average vascular diameter produced by 10⁻⁷ M PGF2ₐ to the eyecup in the presence of the peptide compared to maximal constriction observed in the absence of the peptide.
²Percent inhibition produced by each peptide is calculated as (100-percent vasomotor response).

Capital letters indicate L-amino acids and small letters indicate D-amino acids. I=isoleucine; L=leucine; G=glycine; H=histidine; R=Arginine; D=Aspartic acid; Y=Tyrosine; K=Lysine; A=Alanine; W=Tryptophan; E=Glutamic acid; F=Phenylalanine; Q=Glutamine; N=Aspargine; P=Proline; S=Serine; X=Cyclohexyl alanine. Peptides were dissolved in DMSO freshly just before the experiment as 10 mM stocks and added to the eye cups 30 min before the addition of 10⁻⁷ M PGF_{2α}.

A total of 25 variants of PCP-8 were synthesized and the efficacious or potent peptides are listed in Table 4. These peptides incorporate L- to D-amino acid changes, deletions, subtle variations in aromaticity, hydrogen bond donor status as opposed to ionic interactions and hydrophobicity. These peptides were tested at 100 μM concentration in porcine retinal vasomotricity assay and the results are summarized in Table 4.

The results are summarized as follows:

1. Converting all L-amino acids of PCP-8 to D-amino acids (PCP-13) increased the inhibitory potency dramatically. Removal of N-terminal hydrophobic dipeptide sequence from either PCP-8 (PCP-11) or PCP-10 (PCP-12) resulted in significant reduction in the inhibitory action of the peptides.

2. Glycine to alanine (13.7) does not change the activity of PCP-13, whereas change to proline (13.16), L-alanine (13.8), or deletion of the residue (13.17) entirely resulted in loss of activity. Glycine is an important linker residue separating the HRD motif from the IL hydrophobic sequence.

3. HRD-motif is important for the activity of PCP-13. Alanine substitutions (13.1-13.3) or to change to L-configuration (13.4-13.6) resulted loss of inhibitory activity of PCP-13. Aromaticity of His is more important than the positive charge, since H to F (13.11) or W (13.22) or X (13.24), but not to Y (13.23), did not result in significant reduction of peptide inhibitory potency. Side chain length appears to be more critical in case of D residue than R; changing D to E (13.12) resulted in loss of half of the inhibitory activity whereas R to K (13.13) or to Q (13.18) affected the activity of PCP-13 moderately. D to N (13.14) resulted in moderate loss of activity, whereas a serine substitution (13.19) lead to drastic loss of activity of PCP-13.

4. Deletion of terminal lysine (13.15) or substitution with W (13.9) resulted in complete loss of activity; however, conversion of terminal carboxylate into an amide (13.20 & 13.21) resulted in moderate gain of activity of the peptide inhibitor. Substitution of aromatic residue, Y, with E (13.10) completely abolished the inhibitory potency of PCP-13.

Thus the structure of PCP-13 in D-configuration appears to consist of a N-terminal hydrophobic anchor spaced from the central HRD motif by a glycine residue possibly resulting in a turn conformation of the active peptide; Aromatic and hydrophobic interactions at the carboxy terminus may also add to the potency of PCP-13.

### Example IV

### Testing PCP Peptides in Porcine Uterine Strip of ex vivo Basal Contraction Assay

In ex vivo experiments using porcine uterine strips, the peptides were able to prevent both basal and PGF_{2α}-induced contraction.

Uterine tissues from non-pregnant adult pigs were obtained from a local slaughter house and transported to the laboratory on ice. Uterine myometrial strips of approximately 1 cm in length were set up in organ baths containing Kreb's buffer equilibrated with 21% oxygen at 37° C. as we have described (Potvin, W. et al., 1990, Br. J. Pharmacol. 100:341-347; Varma, D. R. and Chemtob, S., 1993, J. Pharmacol. Expt. Ther. 265:1096-1104). Contractions were recorded by force transducers on Grass-polygraph. Strips were incubated with or without 100 [N peptides for 30 min before adding PGF_{2α} in step-wise increments (10⁻⁹ to 10⁻⁶ M). Data were expressed as percentage increase over the basal level of average tension (g).

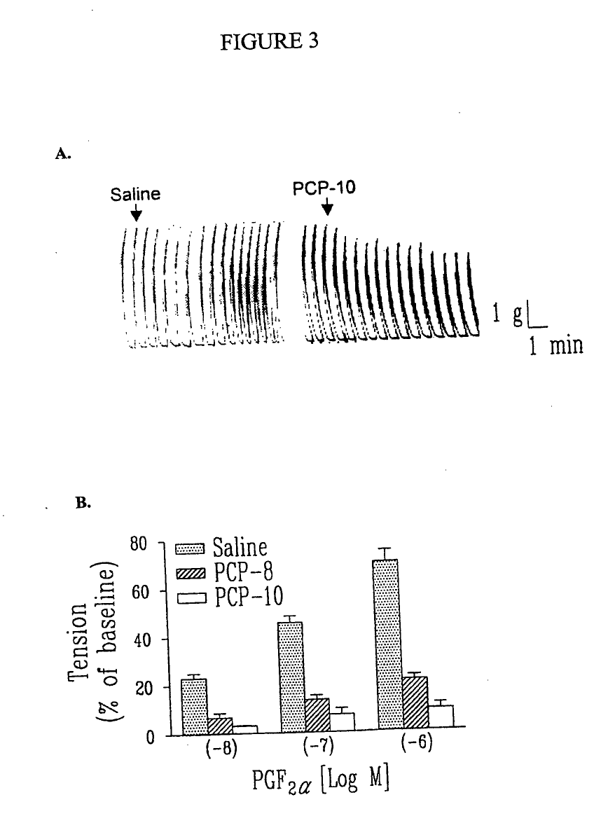

A graph of spontaneous uterine contractions (known to be dependent upon prostanoids, mainly PGF_{2α}) in the absence and the presence of 100 μM PCP-8 are shown in FIG. 3A. Addition of peptide to the strips reduced the force of basal contraction. A dose response of PGF2ₐ on uterine contractility in the presence or absence of PCP-8 and PCP-10 peptides is shown in FIG. 33. More than 600 (PCP-8) and 80% (PCP-10) reduction in uterine contraction was observed in all concentrations of PGF_{2α} tested. Thus, both these peptides reduced spontaneous as well as PGF_{2α}-induced contractions in the uterine strips.

### Example V

### Testing PCP Peptides in Bovine Uterine Strip of ex vivo Basal Contraction Assay

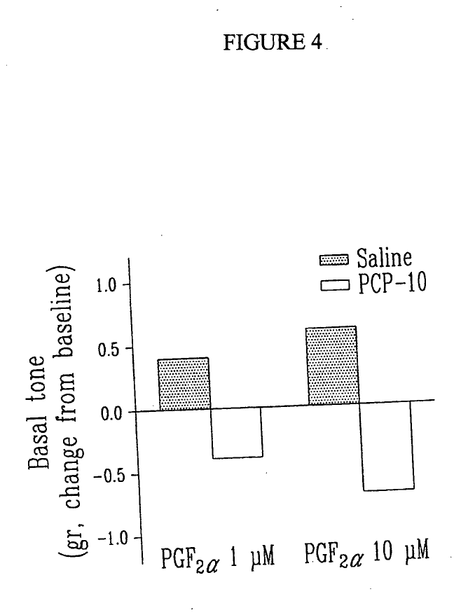

Uterine tissues from non-pregnant adult bovine animals were obtained from a local slaughter house and transported to the laboratory on ice. Uterine myometrial strips of approximately 1 cm in length were set up in organ baths containing Kreb's buffer equilibrated with 21% oxygen at 37° C. as described above. Contractions were recorded on Grass-polygraph by force transducers. Strips were incubated with or without 100 μM peptides before adding PGF_{2α} in step-wise increments (10⁻⁸ to 10⁻⁶ M). Data were expressed as change in basal level of average tension (g). The results are shown in FIG. 4. Application of PCP-10 peptide at 100 μM reversed the basal tone (contractile state) of the uterine muscle. Addition of PGF_{2α} up to 10 μM did not affect the relaxation produced by PCP-10 suggesting that the effects of PCP peptides are independent of the ligand, which was also shown in the previous results.

### Example VI

### Identification of Urotensin (GPR14) Receptor Antagonists in an ex vivo Porcine Retinal Microvascular Contraction Assay

Urotensin receptor (GPR14) peptide antagonists having the following sequences were prepared:

| | |
|---|---|
| NH2-(d)Leu-(d)Ala-(d)Met-(d)Arg-(d)Leu-(d)Val-(d)Arg-(d)Arg-Gly-OH | SEQ ID NO:78 |
| NH2-(d)Val-(d)Ile-(d)Arg-(d)Ala-(d)Thr-(d)Arg-(d)Pro-(d)Ala-(d)Leu-OH | SEQ ID NO:79 |

To prepare eyecups from piglet eyes, a circular incision was made 3-4 mm posterior to ora serrata to remove the interior segment and vitreous body with minimal handling of the retina. The remaining eyecup was fixed with pins to a wax base in a 20 ml tissue bath containing 20 ml of Kreb's buffer (pH 7.35-7.45) and equilibrated with 21% oxygen and 5% carbon dioxide at 37° C. The preparations were allowed to stabilize for 30 min. The outer vessel diameter was recorded with a video camera mounted on a dissecting microscope (Zeiss M 400) and the responses were quantified by a digital image analyzer (Sigma Scan Software, Jandel Scientific, Corte Madera, Calif.). Vascular diameter was recorded before and 5 min following the topical application of the agonist. Each measurement was repeated three times and showed <1% variability. Cumulative concentration-response curves of urotensin-II (U-II) or the antagonists (10⁻¹⁰ to 10⁻⁵ M) were constructed. To assess the reversibility of the antagonists, the eyecups were thoroughly washed (which would wash away the peptide) with incubation medium and concentration response curves for U-II were determined.

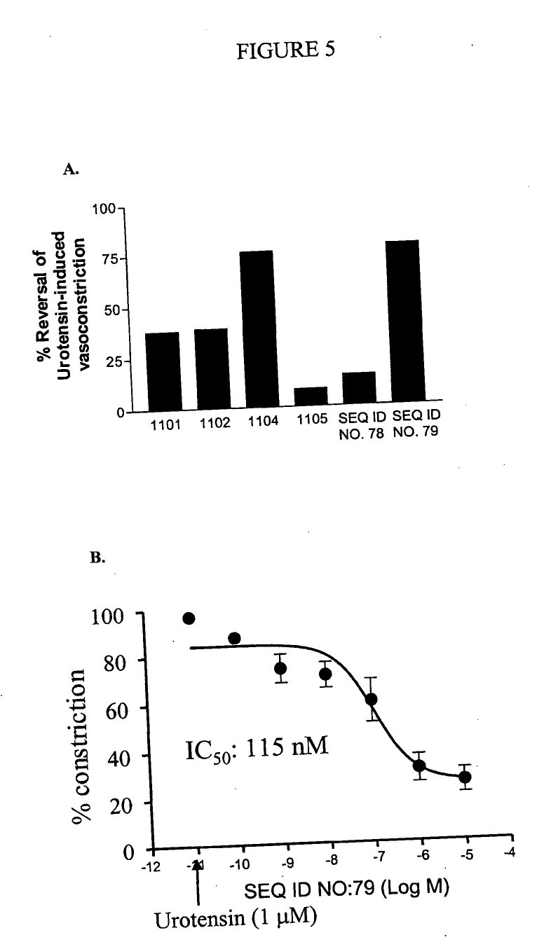

Incubation of the retinal tissues with peptides prior to adding U-II at 1 μM resulted in various degrees of inhibition of contraction by the peptides FIG. 5A; of the six peptides tested, ThG1104 and SEQ ID NO:79 reversed U-II induced contraction by 75% whereas 1101 and 1102 showed moderate inhibition (35-40%); SEQ ID NO:78 and peptide 1106 were least effective (10-15%). Dose-response of SEQ ID NO:79 on U-II (1 μM) mediated contractility of retinal microvessels revealed an IC₅₀ of 115 nM (FIG. 5B)

### Example VII

### Effect of SEQ ID NO:79 on U-II Induced Inositol Phosphate Hydrolysis

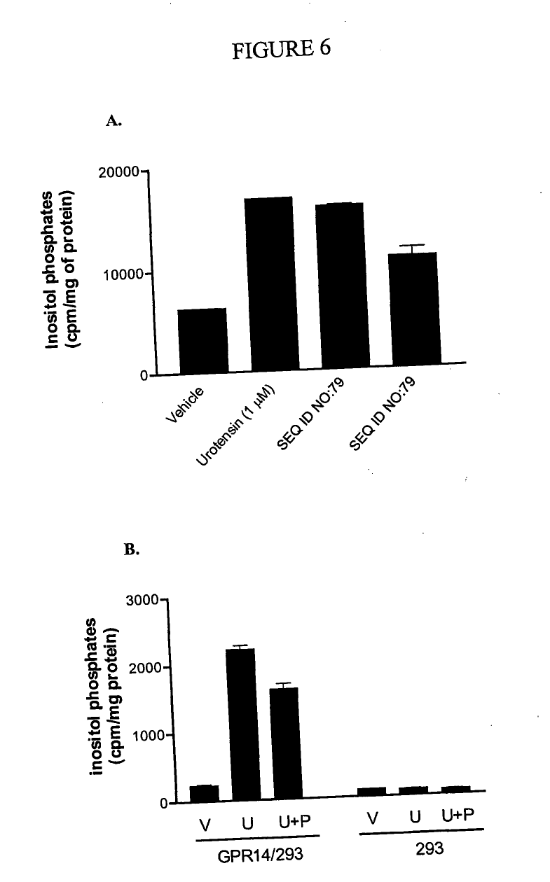

Using the peptide antagonists as described in Example VI, the ability of SEQ ID NO:79 to modulate second messenger (inositol phosphates) levels produced in response to U-II stimulation of its receptor, GPR14, was determined by measuring the levels of phosphoinositides in piglet aortic strips and in HEK293 cells expressing recombinant GPR14. Aortic strips or GPR14/293 cells were labelled with [3H] myoinositol (2 μci/ml medium) overnight and stimulated with 1 μM U-II in the presence/absence of SEQ ID NO:79 (10-100 μM) for 1 h. The total phosphoinositides were collected from tissue/cell homogenates by anion exchange chromatography with 1 M ammonium formate/0.1 N formic acid. SEQ ID NO:79 dose-dependently inhibited U-II stimulated synthesis of total phosphoinositides in aortic strips (FIG. 6A). Similar inhibition of U-II (U) stimulated phosphoinositide hydrolysis by SEQ ID NO:79 (P) was observed in GPR14/293 cells, whereas parent HEK293 cells did not respond to U-II (FIG. 6B); V is vehicle (saline) treated cells.

### Example VII

### Effect of SEQ ID NO:79 on Somatostatin-14 Contractile Responses in Porcine Retinal Microvessels

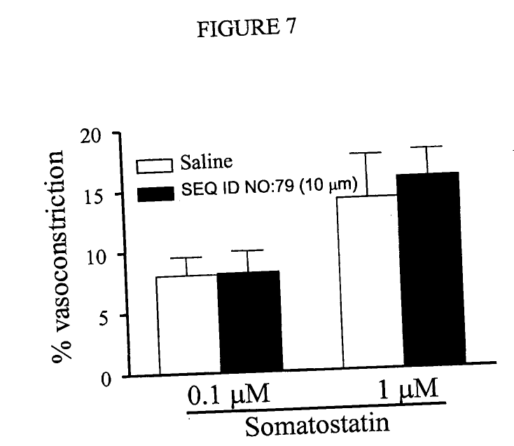

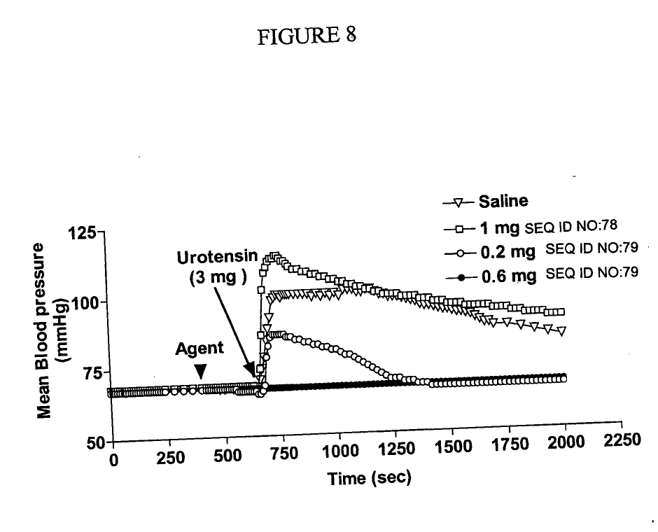

Somatostatin-14 is highly related to U-II in structure and sequence. To find out if the antagonism of SEQ ID NO:79 is restricted to U-II responses, somatostatin-14 effects on microvascular contractility were measured in the presence/absence of 10 μM SEQ ID NO:79. As shown in FIG. 7, somatostatin-14 produced moderate contraction in retinal microvasculature; SEQ ID NO:79 did not antagonize somatostatin effects.

### Example VIII

### Effect of SEQ ID NO:79 on U-II Induced Acute Hypertension in Piglet

Newborn pigs (1-3 days old: 1-1.5 Kg) were anesthetized with 1.5% halothane for tracheostomy and catheterization of the right femoral vein for drug administration. Animals were ventilated by means of a Harvard small animal respirator with a gas mixture of 25% O₂ and 75% N₂. Halothane was discontinued after surgery and immediately thereafter the animals were sedated with □-chloralose (50 mg/kg i.v.) and paralyzed with pancuronium (0.1 mg/kg i.v.). Animals were placed under radiant warmer to keep their body temperature at 37° C. Mean arterial pressure was monitored via carotid catheter connected to Gould pressure transducers and a polygraph. U-II (3 mg bolus i.v.) produced significant hypertension in piglet; SEQ ID NO:78 (1 mg bolus i.v.), which did not block U-II induced microvascular effects in ex vivo assays, also was ineffective in counteracting U-II induced hypertension. However, there was a potent dose-dependent blockade of U-II induced acute hypertension by SEQ ID NO:79; at 0.6 mg (i.v. bolus), SEQ ID NO:79 completely abolished U-II effects of mean blood pressure.

### Example IX

### Effect of SEQ ID NO:79 on U-II Induced Acute Constriction in Upper Bronchi

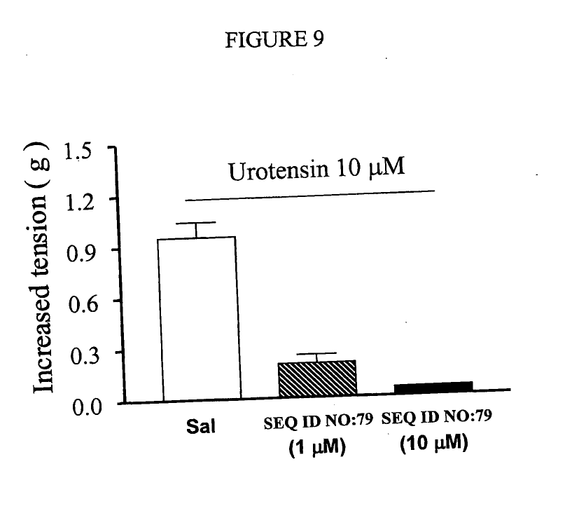

Hay W et al (Br. J. Pharmacol. 2000. 131: 10-12) showed potent bronchoconstriction in primary bronchi by U-II and suggested that U-II may play a role in tracheobronchial contractility. In order to find out if SEQ ID NO:79 could antagonize U-II responses in primary bronchi, the bronchial rings (3 mm in diameter) were suspended in organ baths at a basal tension of 1.5 g. Contractile responses were recorded using Gould pressure transducers connected to a polygraph. As shown in FIG. 9, SEQ ID NO:79 potently blocked U-II induced contraction in a dose-dependent manner.

### Example X

### Testing Peptide Antagonists of the Glucagon Receptor in a Rat Model of Glucagon-Induced Hyperglycemia

Glucagon receptor peptide antagonists having the following sequences were prepared:

| | |
|---|---|
| NH2-(d)Leu-(d)Val-(d)Ile-(d)Asp-Gly-(d)Leu-(d)Leu-(d)Arg-(d) Thr-OH | SEQ ID NO:80 |
| NH2-(d)Ala-(d)Ala-(d)Val-(d)Arg-(d)Cys-Gly-(d)Ala-(d)Val-OH | SEQ ID NO:81 |
| NH2-(d)Phe-(d)Val-(d)Thr-(d)Asp-(d)Glu-(d)His-(d)Ala-(d)Gln-OH | SEQ ID NO:82 |

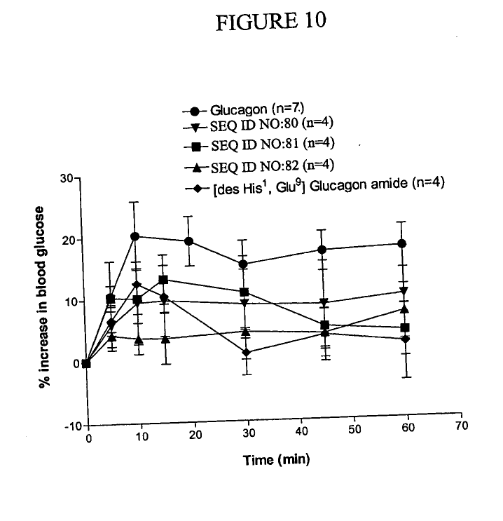

Normal male sprague-Dawley rats (290-320 g) fasted for 4-6 h were sedated with isofluorane and given peptide antagonists in saline via the jugular vein. Glucagon (1-29) amide (4 μg/rat), [des His1], [Glu9] glucagon (1-29) amide, a known glucagon receptor antagonist (10 μg/rat) and SEQ ID NOs:80, 81 and 82 (1 mg/kg) were given i.v. The blood samples were drawn from the carotid artery and the glucose levels measured with a portable glucometer (Lifescan). As shown in FIG. 10, SEQ ID NOs: 80, 81 and 82 blocked glucagon-induced hyperglycemia in this model and this effect was persistent for 60 min.

### Example XI

### Testing Peptide Antagonists of the Glucagon Receptor in a Rat Hepatocyte Assay

Hepatocyte Preparation

All experimental procedures are performed under isoflurane (2.5%) anesthesia according to an experimental protocol approved by the Ste-Justine Hospital Research Center animal care committee. The peptide antagonist represented by SEQ ID NO: 82 was used. Briefly, an incision is made across the abdomen of the animal to reveal the liver and allow access to the superior vena cava. The animal is perfused with a perfusion buffer (in mM: glucose, 20; bicarbonate, 25; Na₂HPO₄, 0.33; NaCl, 136.7; KH₂PO₄ 0.44, KCl, 5.4; Heparin, 5U/ml; pH 7.4) through the heart to remove a maximal amount of blood from the liver (until it acquires a light brown color). A catheter (PE-90) is then inserted in the portal vein and the liver further perfused to eliminate any trace of blood. The hepatic artery is also cannulated (PE-50) and perfused. The liver is then carefully removed from the abdominal cavity and placed into a 250 ml beaker containing HEPES buffer at 37° C. (in mM: HEPES, 25; NaCl, 18.1; KCl, 5; CaCl₂, 1.5; MgCl₂, 1; dextrose, 20; pH 7.4). Collagenase (9650 U) and elastase (20 U) are added to the buffer and circulated via the catheters in a closed loop for 10 minutes. The buffer is replaced with a fresh solution, fresh collagenase and elastase added, and perfusion continued for an additional 10 minutes. The liver is removed and the hepatocytes dissociated by gentle shaking until the liver tissue is pasty in appearance. The cells are filtered with a tea strainer and centrifuged at 52×g for 3 minutes. The cells are resuspended and washed two more times. Typically the yield is 120 to 160 million viable hepatocytes from 1 liver (300 g rat). The hepatocytes are used within one hour of preparation.

cAMP assay

cAMP stimulation studies are performed in tubes containing 1 million cells each. The cells are treated with 0.1 mM IBMX (0.5 μg per tubes, with or without library peptides, 10−6 M or otherwise indicated) in 250 μl of HEPES buffer for 5 min followed by another 5 minutes of treatment with glucagon (10−7 M). The tubes are frozen and stored at −80° C. The cell pellets are thawed by adding 500 μl of 70% EtOH, vortexing for a few seconds and incubating at 37° C. for 10 min. The tubes are centrifuged at 13,000×g for 10 min at 4° C. and the supernatants lyophilized in a speed-vac. The cAMP levels in the tubes are determined using a radioimmunoassay kit (Amersham DPC kit). Des [His1] Glu9 glucagon amide (10−6 M) is used as a control. The data are expressed as pmol cAMP/million cells.

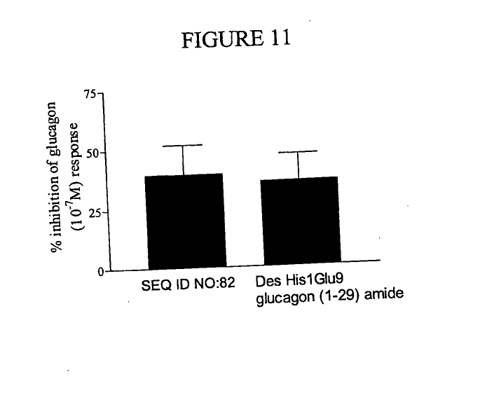

As shown in FIG. 11, both SEQ ID NO:82 (n=4) and known glucagon receptor antagonist, des His1 glu9 glucagon (1-29) amide (n=4) produced comparable inhibition of glucagon-induced cAMP synthesis in rat primary hepatocytes.

### Example XII

### Identification and Characterization of Peptide Inhibitors of R-14 Receptor

R14 orphan receptor antagonists having the following sequences were prepared:

| | |
|---|---|
| NH2-(d)His-(d)Ile-(d)Ile-(d)Cys-(d)Ser-(d)Pro-(d)Leu-(d)Arg-OH | SEQ ID NO:83 |
| NH2-(d)Ile-(d)Phe-(d)Phe-(d)Asp-(d)Ser-(d)Thr-(d)Glu-(d)Cys-(d)Trp-OH | SEQ ID NO:84 |

Newborn pigs (1-3 days old) were anesthetized with 1.5% halothane for tracheostomy and catheterization of the right femoral vein for drug administration. Animals were ventilated by means of a Harvard small animal respirator with a gas mixture of 25% O₂ and 75% N₂. Halothane was discontinued after surgery and immediately thereafter the animals were sedated with α-chloralose (50 mg/kg i.v.) and paralyzed with pancuronium (0.1 mg/kg i.v.). Animals were placed under radiant warmer to keep their body temperature at 37° C. A butterfly needle (24G) is inserted into the anterior chamber of both eyes and connected to a Statham pressure transducer connected to a Gould multichannel recorder. Intraocular pressure is then allowed to stabilize for 15 minutes. Peptide antagonists dissolved in saline were topically applied as drops under the lower eyelid and allowed to diffuse. The intraocular pressure was monitored for 15 minutes at which time a maximal effect was observed.

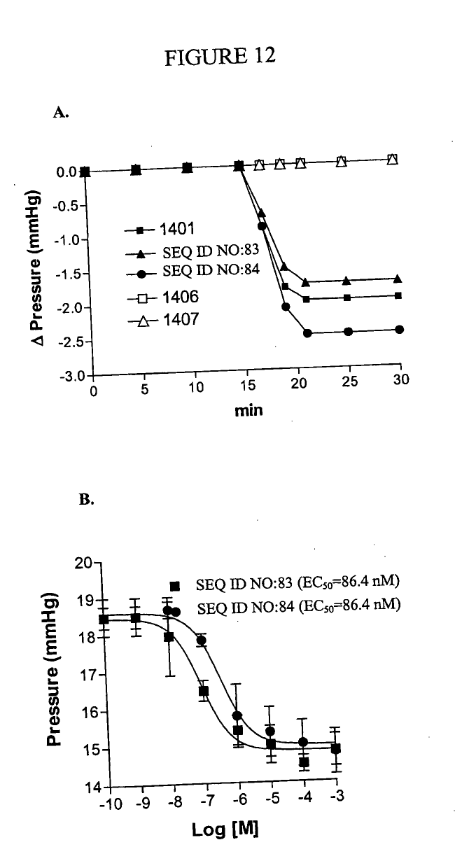

FIG. 12(A) shows the effects of peptide antagonists on intraocular pressure in piglet. Peptide antagonists dissolved in saline were applied to the eye after 15 min of stabilization of intraocular pressure and changes in pressure were monitored for 15 min. The deviation (negative for hypotension) was plotted as a function of time (min). Of the peptides tested, 1401, SEQ ID NO:83 and SEQ ID NO:84 produced a decrease in ocular pressure within minutes of application, whereas 1406 and 1407 did not affect the basal IOP in the animals.

FIG. 12(B) shows the dose-response of SEQ ID NO:83 and SEQ ID NO:84 on intra-ocular pressure in piglets. The experiments were performed as above, except that intraocular pressure was plotted versus the dose of peptide used. Dose response of the peptides on basal IOP of piglet revealed IC₅₀ values of 86.4 and 341.6 nM for SEQ ID NO:83 and SEQ ID NO:84 respectively.

### Example XIII

### Comparison of the Efficacy of SEQ ID NO:84 with Latanoprost and Timolol

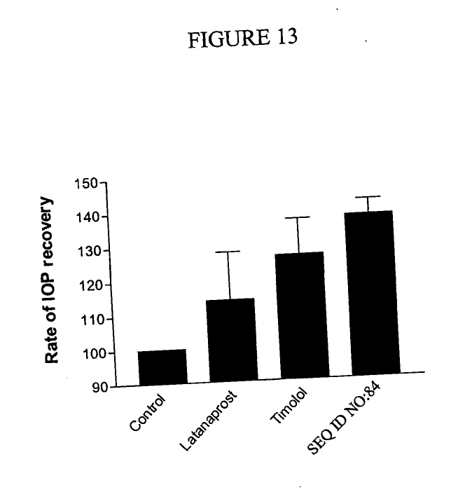

Immediately after euthanasia, rabbit eyeballs were collected and placed into 15-ml organ baths (Radnoti Glass, Monrovia, Calif.). Baths were filled with Krebs (composition in mM: NaCl 120, KCl 4.5, CaCl₂ 2.5, MgSO₄ 1.0, NaHCO₃ 27, KH₂PO₄ 1.0, and glucose 10, pH 7.4), maintained at 37° C. and bubbled with 95% O₂. The eyeballs were maintained in place with the cornea facing upwards. A butterfly needle (24G) is inserted into the anterior chamber of the eye and connected to a Statham pressure transducer connected to a Gould multichannel recorder. Intraocular pressure is then increased to 35 mmHg manually with saline. The recovery rate (return to 20 mmHg) in the absence (control) or presence of topically applied agents was assessed. Two measurements were averaged, both in the relatively linear portion of the recovery. Control measurements were repeated twice, giving identical results. All values were obtained on the same rabbit eye. Ophthalmic preparations of latanoprost (0.03%) and timolol (0.5%) in addition to SEQ ID NO:84 (0.1%) in saline were applied as drops to the eyes. As shown in FIG. 13, SEQ ID NO:84 reversed experimentally-induced ocular hypertension with an efficiency similar to that of timolol, but significantly faster than latanoprost.

### Example XIV

### Selectivity of SEQ ID NO:83 and SEQ ID NO:84

Adult pig eyecup preparations were used to study the response in situ of the relatively undisturbed retinal vasculature. Briefly, a circular incision was made 3-4 mm posterior to the ora serrata to remove the anterior segment and vitreous body with minimal handling of the retina. The remaining eyecup was fixed with pins to a wax base in a 20-ml tissue bath containing Krebs buffer (pH 7.35-7.45) equilibrated with 21% O₂ and 5% CO₂ and maintained at 37° C. The preparations were allowed to stabilize for 30-45 min, during which they were rinsed two or three times with fresh buffer. Cumulative concentration-response curves to different agents were constructed separately on non-perfused primary arterioles (100-200 μm diameter) of fresh tissue. The outer vessel diameter was recorded with a video camera mounted on a dissecting microscope (model M-400, Zeiss), and responses were quantified by a digital image analyzer (Sigma Scan software, Jandel Scientific, Corte Madera, Calif.). Vascular diameter was recorded before and 10 min after topical application of each concentration of agent, at which time a stable response was generally achieved. Each measurement was repeated three times, and variability was <1%. Additional experiments can be performed after a 20 min pretreatment with a variety of blocking or modulating agents. The responses are expressed as percent change in the outer diameter of vessel from baseline or as a percent reversal of a constrictor agent (Thromboxane receptor agonist, U46619 at a concentration producing a 70% of its maximal effects).

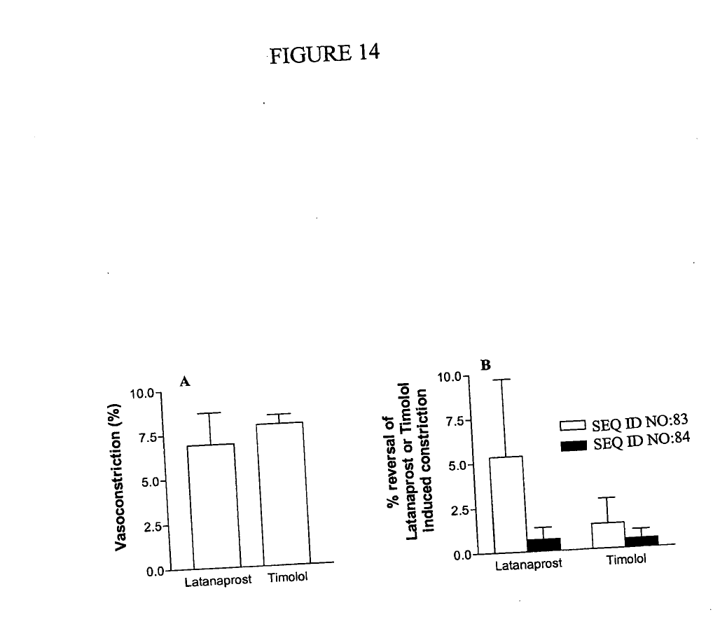

Both latanoprost and timolol constricted the porcine adult retinal arterioles by an average of 7-8% (FIG. 14A). The R-14 peptide antagonists, SEQ ID NOs:83 and 84 were tested to measure the relative selectivity of these compounds compared to known ocular hypotensive compounds, latanoprost and timolol. As shown in FIG. 14(B), both SEQ ID NOs:83 and 84 did not reverse the constriction produced by latanoprost (by 1-5%) or by timolol (<1%).

### Example XV

### Testing PAF Receptor Antagonist Peptide in Porcine Vasomotor Assay

A platelet activating factor (PAF) receptor peptide antagonist having the following sequence was prepared:

| | |
|---|---|
| NH2-(d)Leu (d)Val (d)Pro (d)Val (d)Ser Gly (d)Lys (d)Glu (d)Tyr-OH | SEQ ID NO:85 |

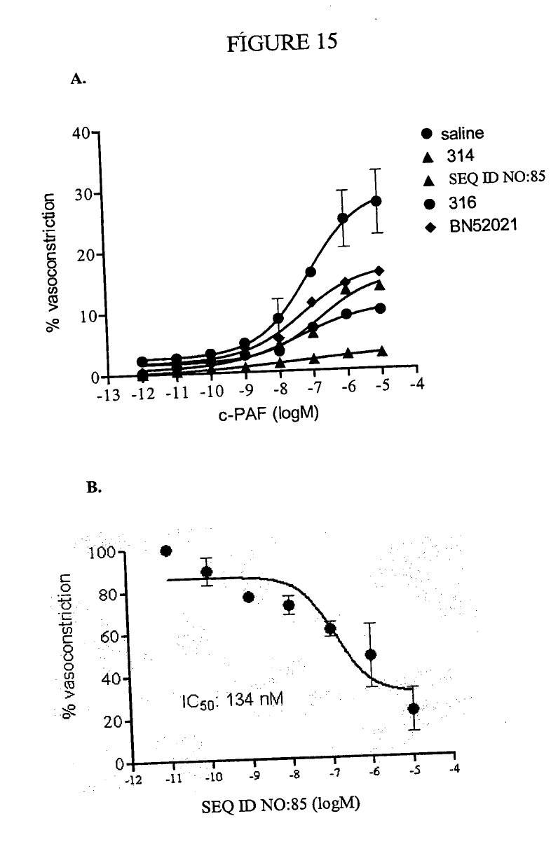

The assay was conducted as described in Example VI. Dose-response curves of C-PAF induced retinal vasomotor responses for PAF receptor antagonists, 314, SEQ ID NO:85 and 316 (peptide antagonists) and BN52021 (small molecule antagonist) are shown in FIGS. 15A and B. SEQ ID NO:85 (1 μM) potently inhibited C-PAF responses even at high concentrations of ligand with an IC₅₀ of 134 nM.

### Example XVI

### Effect of SEQ ID NO:85 on C-PAF Induced Hypotension in Piglet

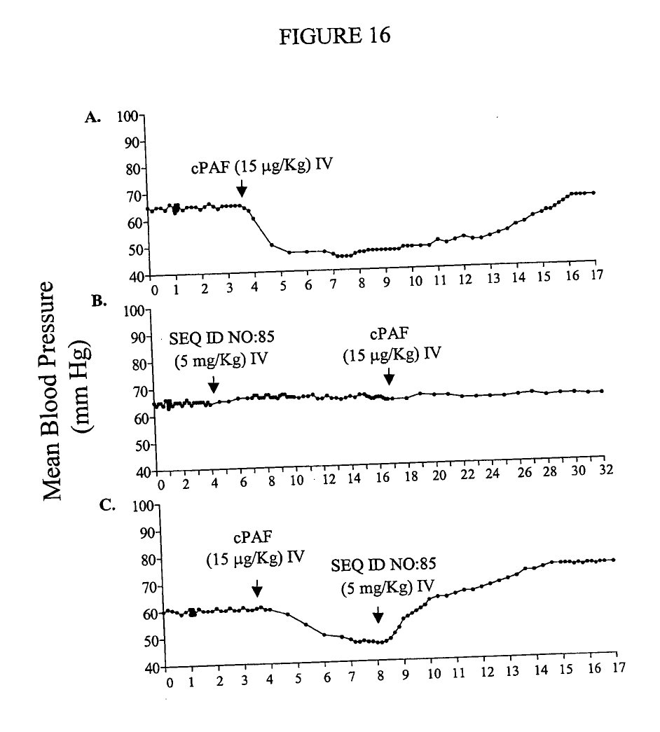

Anesthetized piglets were catheterized to monitor mean arterial pressure and to administer compounds as described in Example VIII. Administration of C-PAF (15 mcg/Kg i.v.) produced immediate drop in blood pressure (FIG. 16A). Pretreatment with SEQ ID NO:85 (1 mg/Kg i.v.) prevented completely C-PAF effect on blood pressure (FIG. 16B). Giving SEQ ID NO:85 (1 mg/kg i.v.) at the nadir of mean arterial pressure produced by C-PAF resulted in quicker recovery of blood pressure (FIG. 16C). These in vivo data demonstrate the inhibitory effect of SEQ ID NO:85 on vascular PAF receptor and its influence on blood pressure which is a cardinal feature of septic shock.

### Example XVII

### Effect of SEQ ID NO:85 on Piglet Model of Septic Shock

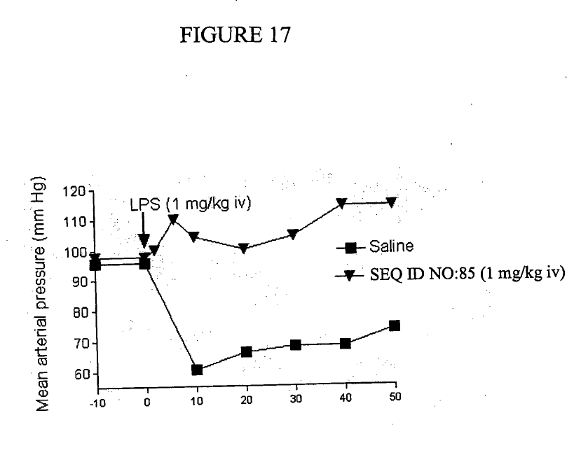

In order to show the utility of SEQ ID NO:85 as a therapeutic in septic shock, a piglet model in which bacterial lipopolysaccharide (LPS) is known to produce hypotension was used. Anesthetized piglets were given LPS (1 mg/kg iv) to produce drastic hypotension (Saline in FIG. 17). Pretreatment with SEQ ID NO:85 (1 mg/kg iv) blocked LPS-induced hypotension completely, thus showing (a) a major initiator of sepsis associated hypotension is PAF and (b) SEQ ID NO:85, a peptide antagonist of PAF receptor could block LPS-induced hypotension completely.

### Example XVIII

### Effect of SEQ ID NO:85 on PAF Receptor Induced Inositol Triphosphate (IP3) Synthesis in Cloned PAF-Receptor Expressing Chinese Hamster Ovary (CHO) Cells

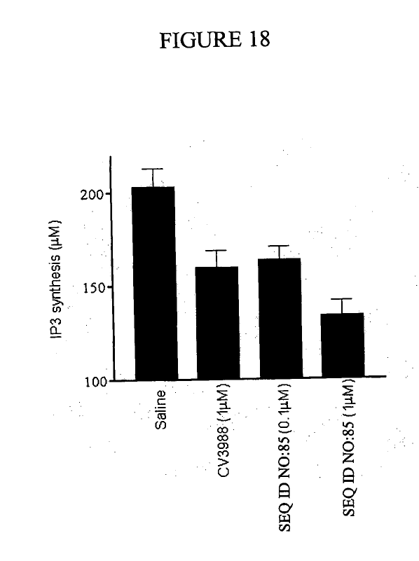

Cell culture and treatment of cells with ligands were carried out using methods outlined in Example VII. IP3 was fractionated using ion exchange columns and expressed as μM per well. As shown in FIG. 18, SEQ ID NO:85 (0.1 μM) inhibited C-PAF (1 μM) induced IP3 synthesis (total indicated in bar-Saline) which was comparable to 1 μM CV3988, a PAF based small molecule competitive antagonist. The inhibition by SEQ ID NO:85 at 1 μM was even more significant than that of same concentration of CV3988. CHO cells devoid of PAF receptor did not respond to either C-PAF or SEQ ID NO:85. Hence SEQ ID NO:85 antagonized PAF actions on CHO cells expressing cloned human PAF receptor, thus demonstrating a receptor-inhibitor interaction.

### Example XIX

### Testing Peptide Antagonists of Parathyroid Hormone Receptor 1 in PTH(1-34) Stimulated cAMP Assay Using ROS Cells

A peptide antagonist to the parathyroid hormone receptor 1 (PTHR1) having the following sequence was prepared:

| | |
|---|---|
| NH2-(d)Phe-(d)Leu-(d)Met-(d)Glu-(d)Tyr-(d)His-(d)Met-(d)Gln-OH | SEQ ID NO:86 |

ROS (rat osteosarcoma) cells are propagated according to supplier's instructions (ATCC). Prior to the experiment, cells were plated in 96-well plates at a density of 80,000 cells/well. After 48 hours of incubation in the presence of 2.5 uM dexamethasone, the cells were first treated with 0.5 mM isobutylmethylxanthine (IBMX) for 10 min followed by 100 nM PTH(1-34) in the presence or absence of SEQ ID NO:86 (100 nM-10 μM). The cAMP levels in the supernatants were determined using a commericial radioimmunoassay kit (DPC kit, Amersham). The data were analysed using GraphPad Prism version 3.02 for Windows (GraphPad Software, San Diego, Calif.).

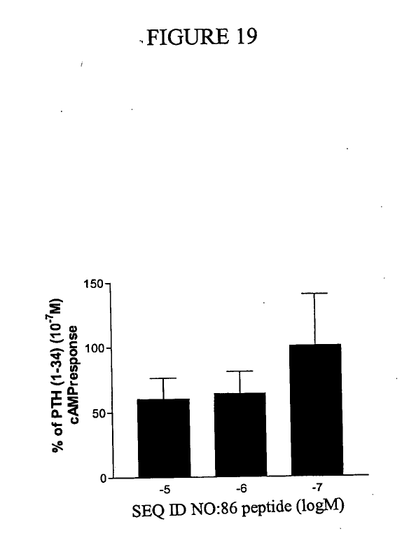

As shown in FIG. 19, SEQ ID NO:86 dose-dependently inhibited the total cAMP produced by 0.1 μM PTH (1-34) in ROS cells.

### Example XX

### Testing Endothelin Receptor Subtype A Antagonists in Pig Retinal Microvascular Contraction Assay

Endothelin receptor subtype A (ET-A) peptide antagonists having the following sequences were prepared:

| | |
|---|---|
| NH2-(d)Phe-(d)Leu-(d)Lys-(d)Cys-(d)Leu-(d)Phe-(d)Val-Gly-OH | SEQ ID NO:87 |
| NH2-(d)Trp-(d)Trp-(d)Asp-(d)Lys-(d)Val-(d)Asp-(d)Gln-(d)Tyr-OH | SEQ ID NO:88 |
| NH2-(d)Glu-(d)Asn-(d)Tyr-(d)Val-(d)Thr-(d)Lys-(d)Lys-(d)Leu-OH | SEQ ID NO:89 |

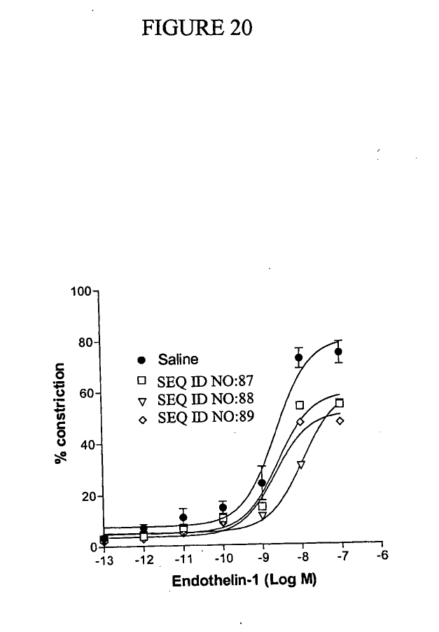

The assay was conducted as described in Example VI. The peptide antagonists were added at 50 μM. Dose responses of endothelin-1 were measured and are shown in FIG. 20. The contractile responses produced by endothelin-1 were inhibited by SEQ ID NOs:87, 88 and 89.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A method of modulating the function of a G-protein coupled receptor (GPCR) in a mammal comprising administering to said mammal an effective amount of at least one GPCR antagonist, wherein said antagonist is a peptide, peptide derivative, peptide analogue or peptidomimetic compound comprising an amino acid sequence of about 5 to about 10 amino acids, said amino acid sequence having at least 70% identity to the sequence of a juxtamembrane extracellular region of said GPCR.

2. The method according to claim 1, wherein said GPCR is selected from the group of: Class A peptide receptors, Class A amide receptors, Class A prostanoid receptors, Class A nucleotide-like receptors, Class A PAF receptors, Class B GPCRs, Class C GPCRs and orphan GPCRs.

3. The method according to claim 2, wherein said GPCR is selected from the group of: Class A peptide receptors, Class A PAF receptors, Class B GPCRs and orphan GPCRs.

4. The method according to claim 1, wherein said antagonist is a peptide comprising at least one D-amino acid.

5. The method according to claim 1, wherein said amino acid sequence has at least 80% identity to the sequence of a juxtamembrane extracellular region of said GPCR.

6. The method according to claim 5, wherein said amino acid sequence has at least 90% identity to the sequence of a juxtamembrane extracellular region of said GPCR.

7. The method according to claim 1, wherein said antagonist further comprises one or more additional N-terminal and/ or C-terminal amino acids that do not correspond to the sequence of a juxtamembrane extracellular region of said GPCR.

8. The method according to claim 1, wherein said antagonist comprises general formula I:
X-A-B-Y (I)
wherein
X is either absent or independently selected from the group comprising: one to four amino acids, a R—CO— or a R—O—CO— group, wherein R is an alkyl, heteroalkyl, a heterocyclic ring, a heteroaromatic ring or an aromatic ring;
A is between 2 and 3 hydrophobic or neutral amino acids;
B is between 4 and 8 hydrophilic or neutral amino acids; and
Y is either absent or independently selected from a group comprising: one to four amino acids, an hydroxyl group, Gly-Lys and Gly-Lys-Lys, and wherein the sequence A-B corresponds to the sequence of a juxtamembrane extracellular region of a GPCR.

9. The method according to claim 3, wherein said antagonist comprises at least 3 consecutive amino acids of a sequence selected from the group of: SEQ ID NOs:1 to 163.

10. The method according to claim 9, wherein said antagonist comprises at least 3 consecutive amino acids from any one of SEQ ID NOs:1 to 20, 29, 31, 33, 77 to 102.

11. The method according to claim 10, wherein said antagonist comprises at least 3 consecutive amino acids from any one of SEQ ID NOs:13 to 20, 29, 31, 33, 77 to 89.

12. A method of identifying a peptide antagonist of a mammalian GPCR comprising:
a) culturing cells in which the GPCR is expressed;
b) contacting the cells with a candidate compound, said candidate compound is a peptide, peptide derivative, peptide analogue or peptidomimetic compound comprising an amino acid sequence of about 5 to about 10 amino acids, said amino acid sequence having at least 70% identity to the sequence of a juxtamembrane extracellular region of said GPCR, and
c) measuring at least one cellular and/or physiological consequence of modulation of GPCR function in said cells,
wherein an increase or decrease in said cellular and/or physiological consequence indicates that the candidate compound is a peptide antagonist of the GPCR.

13. The method according to claim 11, wherein said at least one cellular and/or physiological consequence is selected from the group of: GTP binding and/or hydrolysis, cellular calcium levels, phosphoinositide hydrolysis, cellular cAMP levels, adenyl cyclase activation or inhibition, protein kinase A activity, phospholipase C activity, cell growth and/or differentiation, gene expression, smooth muscle contraction or dilation, vasoconstriction or dilation, nerve cell membrane potential and secretion from glandular cells.

14. A method of modulating a cellular or physiological process mediated by a mammalian G-protein coupled receptor (GPCR), said method comprising contacting cells expressing said GPCR with an effective amount of at least one GPCR antagonist, wherein said antagonist is a peptide, peptide derivative, peptide analogue or peptidomimetic compound comprising an amino acid sequence of about 5 to about 10 amino acids, said amino acid sequence having at least 70% identity to the sequence of a juxtamembrane extracellular region of said GPCR.

15. The method according to claim 13, wherein said cells are in nitro and said process is cellular.

16. The method according to claim 13, wherein said cells are in vivo.


SEQUENCE LISTING (163 sequences)

SEQ ID NO: 1 - protein, 8 aa, Artificial Sequence
  peptide derived from the sequence of Prostaglandin F2-alpha Receptor
ILGHRDYK

SEQ ID NO: 2 - protein, 8 aa, Artificial Sequence
  peptide derived from the sequence of Prostaglandin F2-alpha Receptor
WEDRFYLL

SEQ ID NO: 3 - protein, 8 aa, Artificial Sequence
  peptide derived from the sequence of Prostaglandin F2-alpha Receptor
YQDRFYLL

SEQ ID NO: 4 - protein, 8 aa, Artificial Sequence
  peptide derived from the sequence of Prostaglandin F2-alpha Receptor
ILAHRDYK

SEQ ID NO: 5 - protein, 8 aa, Artificial Sequence
  peptide derived from the sequence of Prostaglandin F2-alpha Receptor
ILGFRDYK

SEQ ID NO: 6 - protein, 8 aa, Artificial Sequence
  peptide derived from the sequence of Prostaglandin F2-alpha Receptor
ILGHKDYK

SEQ ID NO: 7 - protein, 8 aa, Artificial Sequence
  peptide derived from the sequence of Prostaglandin F2-alpha Receptor
ILGHRNYK

SEQ ID NO: 8 - protein, 8 aa, Artificial Sequence
  peptide derived from the sequence of Prostaglandin F2-alpha Receptor
ILGHQDYK

SEQ ID NO: 9 - protein, 7 aa, Artificial Sequence
  peptide derived from the sequence of Prostaglandin F2-alpha Receptor
ILGHRDY

SEQ ID NO: 10 - protein, 8 aa, Artificial Sequence
  peptide derived from the sequence of Prostaglandin F2-alpha Receptor
ILGWRDYK

SEQ ID NO: 11 - protein, 8 aa, Artificial Sequence
  peptide derived from the sequence of Prostaglandin F2-alpha Receptor
ILGXRDYK

SEQ ID NO: 12 - protein, 8 aa, Artificial Sequence
  peptide derived from the sequence of Prostaglandin F2-alpha Receptor
SNVLCSIF

SEQ ID NO: 13 - protein, 9 aa, Artificial Sequence
  peptide derived from the sequence of Urotensin Receptor
LAMRLVRRG

SEQ ID NO: 14 - protein, 9 aa, Artificial Sequence
  peptide derived from the sequence of Urotensin Receptor
VIRATRPAL

SEQ ID NO: 15 - protein, 9 aa, Artificial Sequence
  peptide derived from the sequence of Glucagon Receptor
LVIDGLLRT

SEQ ID NO: 16 - protein, 8 aa, Artificial Sequence
  peptide derived from the sequence of Glucagon Receptor
AAVRCGAV

SEQ ID NO: 17 - protein, 8 aa, Artificial Sequence
  peptide derived from the sequence of Glucagon Receptor
FVTDEHAQ

SEQ ID NO: 18 - protein, 8 aa, Artificial Sequence
  peptide derived from the sequence of R14 Orphan Receptor (AAL86878)
HIICSPLR

SEQ ID NO: 19 - protein, 9 aa, Artificial Sequence
  peptide derived from the sequence of R14 Orphan Receptor (AAL86878)
IFFDSTECW

SEQ ID NO: 20 - protein, 9 aa, Artificial Sequence
  peptide derived from the sequence of Platelet Activating Factor (PAF)Receptor
LVPVSGKEY

SEQ ID NO: 21 - protein, 8 aa, Artificial Sequence
  peptide derived from the sequence of Platelet Activating Factor (PAF) Receptor
FLTYRFES

SEQ ID NO: 22 - protein, 8 aa, Artificial Sequence
  peptide derived from the sequence of Platelet Activating Factor (PAF) Receptor
NGQNQYYV

SEQ ID NO: 23 - protein, 8 aa, Artificial Sequence
  peptide derived from the sequence of Platelet Activating Factor (PAF)Receptor
GAVNCLFK

SEQ ID NO: 24 - protein, 8 aa, Artificial Sequence
  peptide derived from the sequence of Platelet Activating Factor (PAF)Receptor
VTNTSDLV

SEQ ID NO: 25 - protein, 8 aa, Artificial Sequence
  peptide derived from the sequence of Platelet Activating Factor (PAF)Receptor
LEALTWPL

SEQ ID NO: 26 - protein, 9 aa, Artificial Sequence
  peptide derived from the sequence of Platelet Activating Factor (PAF)Receptor
LCLTVQHAD

SEQ ID NO: 27 - protein, 8 aa, Artificial Sequence
  peptide derived from the sequence of Endothelin (ETA) Receptor
NIYKFAST

SEQ ID NO: 28 - protein, 10 aa, Artificial Sequence
  peptide derived from the sequence of Endothelin (ETA) Receptor
KFVNIPLDIV

SEQ ID NO: 29 - protein, 8 aa, Artificial Sequence
  peptide derived from the sequence of Endothelin (ETA) Receptor
FLKCLFVG

SEQ ID NO: 30 - protein, 9 aa, Artificial Sequence
  peptide derived from the sequence of Endothelin (ETA) Receptor
HQEGRYEFL

SEQ ID NO: 31 - protein, 8 aa, Artificial Sequence
  peptide derived from the sequence of Endothelin (ETA) Receptor
WWDKVDQY

SEQ ID NO: 32 - protein, 9 aa, Artificial Sequence
  peptide derived from the sequence of Endothelin (ETA) Receptor
ENYVTKKLV

SEQ ID NO: 33 - protein, 8 aa, Artificial Sequence
  peptide derived from the sequence of Endothelin (ETA) Receptor
ENYVTKKL

SEQ ID NO: 34 - protein, 9 aa, Artificial Sequence
  peptide derived from the sequence of Endothelin (ETA) Receptor
LLECRENKD

SEQ ID NO: 35 - protein, 10 aa, Artificial Sequence
  peptide derived from the sequence of alpha-1A Adrenergic Receptor (AR)
VNISKAILLG

SEQ ID NO: 36 - protein, 12 aa, Artificial Sequence
  peptide derived from the sequence of alpha-1A Adrenergic Receptor (AR)
VLGYWAFGRVFC

SEQ ID NO: 37 - protein, 12 aa, Artificial Sequence
  peptide derived from the sequence of alpha-1A Adrenergic Receptor (AR)
YWAFGRVFCNIW

SEQ ID NO: 38 - protein, 12 aa, Artificial Sequence
  peptide derived from the sequence of alpha-1A Adrenergic Receptor (AR)
FGWRQPAPEDET

SEQ ID NO: 39 - protein, 12 aa, Artificial Sequence
  peptide derived from the sequence of alpha-1A Adrenergic Receptor (AR)
ICQINEEPGYVL

SEQ ID NO: 40 - protein, 12 aa, Artificial Sequence
  peptide derived from the sequence of alpha-1A Adrenergic Receptor (AR)
FFPDFKPSETVF

SEQ ID NO: 41 - protein, 12 aa, Artificial Sequence
  peptide derived from the sequence of alpha-1A Adrenergic Receptor (AR)
FFPDFKPSETVF

SEQ ID NO: 42 - protein, 12 aa, Artificial Sequence
  peptide derived from the sequence of 5H1A Serotonin Receptor (SR)
GISDVTVSYQVI

SEQ ID NO: 43 - protein, 12 aa, Artificial Sequence
  peptide derived from the sequence of 5H1A Serotonin Receptor (SR)
VLNKWTLGQVTC

SEQ ID NO: 44 - protein, 12 aa, Artificial Sequence
  peptide derived from the sequence of 5H1A Serotonin Receptor (SR)
KWTLGQVTCDLF

SEQ ID NO: 45 - protein, 12 aa, Artificial Sequence
  peptide derived from the sequence of 5H1A Serotonin Receptor (SR)
LGWRTPEDRSDP

SEQ ID NO: 46 - protein, 12 aa, Artificial Sequence
  peptide derived from the sequence of 5H1A Serotonin Receptor (SR)
CTISKDHGYTIY

SEQ ID NO: 47 - protein, 12 aa, Artificial Sequence
  peptide derived from the sequence of 5H1A Serotonin Receptor (SR)
VLPFCESSCHMP

SEQ ID NO: 48 - protein, 12 aa, Artificial Sequence
  peptide derived from the sequence of 5H1A Serotonin Receptor (SR)
FCESSCHMPTLL

SEQ ID NO: 49 - protein, 12 aa, Artificial Sequence
  peptide derived from the sequence of Adenosine A1 Receptor
PPSISAFQAAYI

SEQ ID NO: 50 - protein, 12 aa, Artificial Sequence
  peptide derived from the sequence of Adenosine A1 Receptor
LINIGPQTYFHT

SEQ ID NO: 51 - protein, 12 aa, Artificial Sequence
  peptide derived from the sequence of Adenosine A1 Receptor
IGPQTYFHTCLM

SEQ ID NO: 52 - protein, 11 aa, Artificial Sequence
  peptide derived from the sequence of Adenosine A1 Receptor
WNNLSAVERAV

SEQ ID NO: 53 - protein, 12 aa, Artificial Sequence
  peptide derived from the sequence of Adenosine A1 Receptor
EPVIKCEFEKVI

SEQ ID NO: 54 - protein, 12 aa, Artificial Sequence
  peptide derived from the sequence of Adenosine A1 Receptor
LFCPSCHKPSIL

SEQ ID NO: 55 - protein, 12 aa, Artificial Sequence
  peptide derived from the sequence of Adenosine A1 Receptor
LFCPSCHKPSIL

SEQ ID NO: 56 - protein, 12 aa, Artificial Sequence
  peptide derived from the sequence of Melanocortin (MC)4 Receptor
GYSDGGCYEQLF

SEQ ID NO: 57 - protein, 12 aa, Artificial Sequence
  peptide derived from the sequence of Melanocortin (MC)4 Receptor
LLNSTDTDAQSF

SEQ ID NO: 58 - protein, 12 aa, Artificial Sequence
  peptide derived from the sequence of Melanocortin (MC)4 Receptor
AQSFTVNIDNVI

SEQ ID NO: 59 - protein, 12 aa, Artificial Sequence
  peptide derived from the sequence of Melanocortin (MC)4 Receptor
IIYSDSSAVIIC

SEQ ID NO: 60 - protein, 12 aa, Artificial Sequence
  peptide derived from the sequence of Melanocortin (MC)4 Receptor
LFIIYSDSSAVI

SEQ ID NO: 61 - protein, 12 aa, Artificial Sequence
  peptide derived from the sequence of Melanocortin (MC)4 Receptor
YISCPQNPYCVC

SEQ ID NO: 62 - protein, 12 aa, Artificial Sequence
  peptide derived from the sequence of Melanocortin (MC)4 Receptor
SCPQNPYCVCFM

SEQ ID NO: 63 - protein, 12 aa, Artificial Sequence
  peptide derived from the sequence of Protease Activated Receptor-2 (PAR-2)
ASVLTGKLTTVF

SEQ ID NO: 64 - protein, 12 aa, Artificial Sequence
  peptide derived from the sequence of Protease Activated Receptor-2 (PAR-2)
FPLKIAYHIHGN

SEQ ID NO: 65 - protein, 12 aa, Artificial Sequence
  peptide derived from the sequence of Protease Activated Receptor-2 (PAR-2)
NWIYGEALCNVL

SEQ ID NO: 66 - protein, 12 aa, Artificial Sequence
  peptide derived from the sequence of Protease Activated Receptor-2 (PAR-2)
LYVVKQTIFIPA

SEQ ID NO: 67 - protein, 12 aa, Artificial Sequence
  peptide derived from the sequence of Protease Activated Receptor-2 (PAR-2)
PEQLLVGDMFNY

SEQ ID NO: 68 - protein, 12 aa, Artificial Sequence
  peptide derived from the sequence of Protease Activated Receptor-2 (PAR-2)
VVHYFLIKSQGQ

SEQ ID NO: 69 - protein, 12 aa, Artificial Sequence
  peptide derived from the sequence of Protease Activated Receptor-2 (PAR-2)
FLIKSQGQSHVY

SEQ ID NO: 70 - protein, 12 aa, Artificial Sequence
  peptide derived from the sequence of Metabotropic Glutamate Receptor 1 (MGR1)
RYLEWSNIEPII

SEQ ID NO: 71 - protein, 13 aa, Artificial Sequence
  peptide derived from the sequence of Metabotropic Glutamate Receptor 1 (MGR1)
LIAKPTTTSCYLQ

SEQ ID NO: 72 - protein, 12 aa, Artificial Sequence
  peptide derived from the sequence of Metabotropic Glutamate Receptor 1 (MGR1)
PTTTSCYLQRLL

SEQ ID NO: 73 - protein, 12 aa, Artificial Sequence
  peptide derived from the sequence of Metabotropic Glutamate Receptor 1 (MGR1)
IIMEPPMPILSY

SEQ ID NO: 74 - protein, 11 aa, Artificial Sequence
  peptide derived from the sequence of Metabotropic Glutamate Receptor 1 (MGR1)
LICNTSNLGVV

SEQ ID NO: 75 - protein, 12 aa, Artificial Sequence
  peptide derived from the sequence of Metabotropic Glutamate Receptor 1 (MGR1)
YFGSNYKIITTC

SEQ ID NO: 76 - protein, 12 aa, Artificial Sequence
  peptide derived from the sequence of Metabotropic Glutamate Receptor 1 (MGR1)
FGSNYKIITTCF

SEQ ID NO: 77 - protein, 8 aa, Artificial Sequence
  peptide derived from the sequence of Parathyroid Hormone (PTH) Receptor
FLMEYHMQ

SEQ ID NO: 78 - protein, 9 aa, Artificial Sequence
  peptide antagonist derived from the sequence of Urotensin Receptor (GPR14)
LAMRLVRRG

SEQ ID NO: 79 - protein, 9 aa, Artificial Sequence
  peptide antagonist derived from the sequence of Urotensin Receptor (GPR14)
VIRATRPAL

SEQ ID NO: 80 - protein, 9 aa, Artificial Sequence
  peptide antagonist derived from the seuqence of Glucagon Receptor
LVIDGLLRT

SEQ ID NO: 81 - protein, 8 aa, Artificial Sequence
  peptide antagonist derived from the sequence of Glucagon Receptor
AAVRCGAV

SEQ ID NO: 82 - protein, 8 aa, Artificial Sequence
  peptide antagonist derived from the sequence of Glucagon Receptor
FVTDEHAQ

SEQ ID NO: 83 - protein, 8 aa, Artificial Sequence
  peptide antagonist derived from the sequence of R14 Orphan Receptor
HIICSPLR

SEQ ID NO: 84 - protein, 9 aa, Artificial Sequence
  peptide antagonist derived from the sequence of R14 Orphan Receptor
IFFDSTECW

SEQ ID NO: 85 - protein, 9 aa, Artificial Sequence
  peptide antagonist derived from the sequence of Platelet Activating Factor (PAF)Receptor
LVPVSGKEY

SEQ ID NO: 86 - protein, 8 aa, Artificial Sequence
  peptide antagonist derived from the sequence of Parathyroid Hormone Receptor 1 (PTHR1)
FLMEYHMQ

SEQ ID NO: 87 - protein, 8 aa, Artificial Sequence
  peptide antagonist derived from the sequence of Endothelin Receptor
FLKCLFVG

SEQ ID NO: 88 - protein, 8 aa, Artificial Sequence
  peptide antagonist derived from the sequence of Endothelin Receptor
WWDKVDQY

SEQ ID NO: 89 - protein, 8 aa, Artificial Sequence
  peptide antagonist derived from the sequence of Endothelin Receptor
ENYVTKKL

SEQ ID NO: 90 - protein, 8 aa, Artificial Sequence
  peptide antagonist based on sequence of prostaglandin F2-alpha receptor
YQDRFYLL

SEQ ID NO: 91 - protein, 8 aa, Artificial Sequence
  peptide antagonist based on sequence of prostaglandin F2-alpha receptor
ILGHRDYK

SEQ ID NO: 92 - protein, 8 aa, Artificial Sequence
  peptide antagonist based on sequence of prostaglandin F2-alpha receptor
ILAHRDYK

SEQ ID NO: 93 - protein, 8 aa, Artificial Sequence
  peptide antagonist based on sequence of prostaglandin F2-alpha receptor
ILAHRDYK

SEQ ID NO: 94 - protein, 8 aa, Artificial Sequence
  peptide antagonist based on sequence of prostaglandin F2-alpha receptor
ILGFRDYK

SEQ ID NO: 95 - protein, 8 aa, Artificial Sequence
  peptide antagonist based on sequence of prostaglandin F2-alpha receptor
ILGHKDYK

SEQ ID NO: 96 - protein, 8 aa, Artificial Sequence
  peptide antagonist based on sequence of prostaglandin F2-alpha receptor
ILGHKNYK

SEQ ID NO: 97 - protein, 8 aa, Artificial Sequence
  peptide antagonist based on sequence of prostaglandin F2-alpha receptor
ILGHQDYK

SEQ ID NO: 98 - protein, 7 aa, Artificial Sequence
  peptide antagonist based on sequence of prostaglandin F2-alpha receptor
ILGHRDY

SEQ ID NO: 99 - protein, 8 aa, Artificial Sequence
  peptide antagonist based on sequence of prostaglandin F2-alpha receptor
ILGHRDYK

SEQ ID NO: 100 - protein, 8 aa, Artificial Sequence
  peptide antagonist based on sequence of prostaglandin F2-alpha receptor
ILGWRDYK

SEQ ID NO: 101 - protein, 8 aa, Artificial Sequence
  peptide antagonist based on sequence of prostaglandin F2-alpha receptor
ILGXRDYK

SEQ ID NO: 102 - protein, 8 aa, Artificial Sequence
  peptide antagonist based on sequence of prostaglandin F2-alpha receptor
SNVLCSIF

SEQ ID NO: 103 - protein, 8 aa, Artificial Sequence
  peptide antagonist based on sequence of prostaglandin F2-alpha receptor
WEDRFYLL

SEQ ID NO: 104 - protein, 8 aa, Artificial Sequence
  peptide antagonist based on sequence of prostaglandin F2-alpha receptor
ILGHRNYK

SEQ ID NO: 105 - protein, 7 aa, Artificial Sequence
  peptide antagonist based on sequence of prostaglandin F2-alpha receptor
ILGHRDY

SEQ ID NO: 106 - protein, 9 aa, Artificial Sequence
  peptide antagonist based on sequence of Urotensin receptor
LAMRLVRRG

SEQ ID NO: 107 - protein, 9 aa, Artificial Sequence
  peptide antagonist based on the sequence of Glucagon Receptor
LVIDGLLRT

SEQ ID NO: 108 - protein, 8 aa, Artificial Sequence
  peptide antagonist based on the sequence of Glucagon Receptor
AAVRCGAV

SEQ ID NO: 109 - protein, 9 aa, Artificial Sequence
  peptide antagonist based on the sequence of Platelet Activating Factor (PAF)Receptor
LVPVSGKEY

SEQ ID NO: 110 - protein, 8 aa, Artificial Sequence
  peptide antagonist based on the sequence of Platelet Activating Factor (PAF)Receptor
FLTYRFES

SEQ ID NO: 111 - protein, 8 aa, Artificial Sequence
  peptide antagonist based on the sequence of Platelet Activating Factor (PAF)Receptor
NGQNQYYV

SEQ ID NO: 112 - protein, 8 aa, Artificial Sequence
  peptide antagonist based on the sequence of Platelet Activating Factor (PAF)Receptor
GAVNCLFK

SEQ ID NO: 113 - protein, 8 aa, Artificial Sequence
  peptide antagonist based on the sequence of Platelet Activating Factor (PAF)Receptor
VTNTSDLV

SEQ ID NO: 114 - protein, 8 aa, Artificial Sequence
  peptide antagonist based on the sequence of Platelet Activating Factor (PAF)Receptor
LEALTWPL

SEQ ID NO: 115 - protein, 9 aa, Artificial Sequence
  peptide antagonist based on the sequence of Platelet Activating Factor (PAF)Receptor
LCLTVQHAD

SEQ ID NO: 116 - protein, 8 aa, Artificial Sequence
  peptide antagonist based on the sequence of Endothelin (ETA) Receptor
NIYKFAST

SEQ ID NO: 117 - protein, 10 aa, Artificial Sequence
  peptide antagonist based on the sequence of Endothelin (ETA) Receptor
KFVNIPLDIV

SEQ ID NO: 118 - protein, 8 aa, Artificial Sequence
  peptide antagonist based on the sequence of Endothelin(ETA)Receptor
FLKCLFVG

SEQ ID NO: 119 - protein, 9 aa, Artificial Sequence
  peptide antagonist based on the sequence of Endothelin(ETA)Receptor
HQEGRYEFL

SEQ ID NO: 120 - protein, 8 aa, Artificial Sequence
  peptide antagonist based on the sequence of Endothelin(ETA)Receptor
ENYVTKKL

SEQ ID NO: 121 - protein, 9 aa, Artificial Sequence
  peptide antagonist based on the sequence of Endothelin (ETA) Receptor
LLECRENKD

SEQ ID NO: 122 - protein, 10 aa, Artificial Sequence
  peptide antagonist based on the sequence of alpha-1A Adrenergic Receptor (AR)
VNISKAILLG

SEQ ID NO: 123 - protein, 12 aa, Artificial Sequence
  peptide antagonist based on the sequence of alpha-1A Adrenergic Receptor (AR)
VLGYWAFGRVFC

SEQ ID NO: 124 - protein, 12 aa, Artificial Sequence
  peptide antagonist based on the sequence of alpha-1A Adrenergic Receptor (AR)
YWAFGRVFCNIW

SEQ ID NO: 125 - protein, 12 aa, Artificial Sequence
  peptide antagonist based on the sequence of alpha-1A Adrenergic Receptor (AR)
FGWRQPAPEDET

SEQ ID NO: 126 - protein, 12 aa, Artificial Sequence
  peptide antagonist based on the sequence of alpha-1A Adrenergic Receptor (AR)
ICQINEEPGYVL

SEQ ID NO: 127 - protein, 12 aa, Artificial Sequence
  peptide antagonist based on the sequence of alpha-1A Adrenergic Receptor (AR)
FFPDFKPSETVF

SEQ ID NO: 128 - protein, 12 aa, Artificial Sequence
  peptide antagonist based on the sequence of alpha-1A Adrenergic Receptor (AR)
FFPDFKPSETVF

SEQ ID NO: 129 - protein, 12 aa, Artificial Sequence
  peptide antagonist based on the sequence of 5H1A Serotonin Receptor (SR)
GISDVTVSYQVI

SEQ ID NO: 130 - protein, 12 aa, Artificial Sequence
  peptide antagonist based on the sequence of 5H1A Serotonin Receptor (SR)
VLNKWTLGQVTC

SEQ ID NO: 131 - protein, 12 aa, Artificial Sequence
  peptide antagonist based on the sequence of 5H1A Serotonin Receptor (SR)
KWTLGQVTCDLF

SEQ ID NO: 132 - protein, 12 aa, Artificial Sequence
  peptide antagonist based on the sequence of 5H1A Serotonin Receptor (SR)
LGWRTPEDRSDP

SEQ ID NO: 133 - protein, 12 aa, Artificial Sequence
  peptide antagonist based on the sequence of 5H1A Serotonin Receptor (SR)
CTISKDHGYTIY

SEQ ID NO: 134 - protein, 12 aa, Artificial Sequence
  peptide antagonist based on the sequence of 5H1A Serotonin Receptor (SR)
VLPFCESSCHMP

SEQ ID NO: 135 - protein, 12 aa, Artificial Sequence
  peptide antagonist based on the sequence of 5H1A Serotonin Receptor (SR)
FCESSCHMPTLL

SEQ ID NO: 136 - protein, 12 aa, Artificial Sequence
  peptide antagonist based on the sequence of Adenosine A1 Receptor
PPSISAFQAAYI

SEQ ID NO: 137 - protein, 12 aa, Artificial Sequence
  peptide antagonist based on the sequence of Adenosine A1 Receptor
LINIGPQTYFHT

SEQ ID NO: 138 - protein, 12 aa, Artificial Sequence
  peptide antagonist based on the sequence of Adenosine A1 Receptor
IGPQTYFHTCLM

SEQ ID NO: 139 - protein, 11 aa, Artificial Sequence
  peptide antagonist based on the sequence of Adenosine A1 Receptor
WNNLSAVERAV

SEQ ID NO: 140 - protein, 12 aa, Artificial Sequence
  peptide antagonist based on the sequence of Adenosine A1 Receptor
EPVIKCEFEKVI

SEQ ID NO: 141 - protein, 12 aa, Artificial Sequence
  peptide antagonist based on the sequence of Adenosine A1 Receptor
LFCPSCHKPSIL

SEQ ID NO: 142 - protein, 12 aa, Artificial Sequence
  peptide antagonist based on the sequence of Adenosine A1 Receptor
LFCPSCHKPSIL

SEQ ID NO: 143 - protein, 12 aa, Artificial Sequence
  peptide antagonist based on the sequence of Melanocortin (MC) 4 Receptor
GYSDGGCYEQLF

SEQ ID NO: 144 - protein, 12 aa, Artificial Sequence
  peptide antagonist based on the sequence of Melanocortin (MC) 4 Receptor
LLNSTDTDAQSF

SEQ ID NO: 145 - protein, 12 aa, Artificial Sequence
  peptide antagonist based on the sequence of Melanocortin (MC) 4 Receptor
AQSFTVNIDNVI

SEQ ID NO: 146 - protein, 12 aa, Artificial Sequence
  peptide antagonist based on the sequence of Melanocortin (MC) 4 Receptor
IIYSDSSAVIIC

SEQ ID NO: 147 - protein, 12 aa, Artificial Sequence
  peptide antagonist based on the sequence of Melanocortin (MC) 4 Receptor
LFIIYSDSSAVI

SEQ ID NO: 148 - protein, 12 aa, Artificial Sequence
  peptide antagonist based on the sequence of Melanocortin (MC) 4 Receptor
YISCPQNPYCVC

SEQ ID NO: 149 - protein, 12 aa, Artificial Sequence
  peptide antagonist based on the sequence of Melanocortin (MC) 4 Receptor
SCPQNPYCVCFM

SEQ ID NO: 150 - protein, 12 aa, Artificial Sequence
  peptide antagonist based on the sequence of Protease Activated Receptor-2 (PAR-2)
ASVLTGKLTTVF

SEQ ID NO: 151 - protein, 12 aa, Artificial Sequence
  peptide antagonist based on the sequence of Protease Activated Receptor-2 (PAR-2)
FPLKIAYHIHGN

SEQ ID NO: 152 - protein, 12 aa, Artificial Sequence
  peptide antagonist based on the sequence of Protease Activated Receptor-2 (PAR-2)
NWIYGEALCNVL

SEQ ID NO: 153 - protein, 12 aa, Artificial Sequence
  peptide antagonist based on the sequence of Protease Activated Receptor-2 (PAR-2)
LYVVKQTIFIPA

SEQ ID NO: 154 - protein, 12 aa, Artificial Sequence
  peptide antagonist based on the sequence of Protease Activated Receptor-2 (PAR-2)
PEQLLVGDMFNY

SEQ ID NO: 155 - protein, 12 aa, Artificial Sequence
  peptide antagonist based on the sequence of Protease Activated Receptor-2 (PAR-2)
VVHYFLIKSQGQ

SEQ ID NO: 156 - protein, 12 aa, Artificial Sequence
  peptide antagonist based on the sequence of Protease Activated Receptor-2 (PAR-2)
FLIKSQGQSHVY

SEQ ID NO: 157 - protein, 12 aa, Artificial Sequence
  peptide antagonist based on the sequence of Metabotropic Glutamate Receptor (MGR1)
RYLEWSNIEPII

SEQ ID NO: 158 - protein, 13 aa, Artificial Sequence
  peptide antagonist based on the sequence of Metabotropic Glutamate Receptor (MGR1)
LIAKPTTTSCYLQ

SEQ ID NO: 159 - protein, 12 aa, Artificial Sequence
  peptide antagonist based on the sequence of Metabotropic Glutamate Receptor (MGR1)
PTTTSCYLQRLL

SEQ ID NO: 160 - protein, 12 aa, Artificial Sequence
  peptide antagonist based on the sequence of Metabotropic Glutamate Receptor (MGR1)
IIMEPPMPILSY

SEQ ID NO: 161 - protein, 11 aa, Artificial Sequence
  peptide antagonist based on the sequence of Metabotropic Glutamate Receptor (MGR1)
LICNTSNLGVV

SEQ ID NO: 162 - protein, 12 aa, Artificial Sequence
  peptide antagonist based on the sequence of Metabotropic Glutamate Receptor (MGR1)
YFGSNYKIITTC

SEQ ID NO: 163 - protein, 12 aa, Artificial Sequence
  peptide antagonist based on the sequence of Metabotropic Glutamate Receptor (MGR1)
FGSNYKIITTCF